(19) United States
(12) Patent Application Publication
(10) Pub. No.: US 2021/0283428 A1
(43) Pub. Date: Sep. 16, 2021

(54) SYSTEMS AND METHODS FOR SIMULTANEOUS MONITORING OF HUMAN NERVE DISPLACEMENT

(71) Applicant: THE TRUSTEES OF COLUMBIA UNIVERSITY IN THE CITY OF NEW YORK, New York, NY (US)

(72) Inventors: Elisa Konofagou, New York, NY (US); Hermes Arytto Salles Kamimura, New York, NY (US); Stephen Alexander Lee, New York, NY (US)

(73) Assignee: THE TRUSTEES OF COLUMBIA UNIVERSITY IN THE CITY OF NEW YORK, New York, NY (US)

(21) Appl. No.: 17/065,230

(22) Filed: Oct. 7, 2020

Related U.S. Application Data
(60) Provisional application No. 62/911,854, filed on Oct. 7, 2019.

(51) Int. Cl. A61N 7/02

(52) U.S. Cl. CPC A61N 7/02; A61N 2007/0026

(57) ABSTRACT

The present subject matter relates to techniques for simultaneous monitoring and modulating target tissue. The disclosed system can include a focused ultrasound (FUS) stimulation probe for stimulating the target tissue, an imaging probe for obtaining ultrasound images of displacement on target tissue, and a processor configured to provide an image of target tissue within about 2 seconds from the stimulating. The imaging probe and the FUS stimulation probe are coaligned and include different center frequencies.

### CROSS-REFERENCE TO RELATED APPLICATION

This application claims priority to U.S. Provisional Patent Application No. 62/911,854, which was filed on Oct. 7, 2019, the entire contents of which are incorporated by reference herein.

### GRANT INFORMATION

This invention was made with government support under grant number HR0011-15-2-0054 awarded by the Defense Advanced Research Projects Agency (DARPA). The government has certain rights in the invention.

### BACKGROUND

Focused ultrasound (FUS) can provide for noninvasive therapeutics and neuromodulation due to its high spatial resolution and deep penetration.

Certain FUS techniques rely upon MRI-based methods or B-mode ultrasound-guided imaging. MRI-guided FUS (MRIgFUS) can be a costly modality, using compliant transducers and equipment to minimize interaction with the magnetic fields. The MRI-compatible transducers can restrict what can be performed inside the coil. Ultrasound-guided (B-mode) FUS does not require specialized equipment. However, B-mode imaging and identification of tissue structures based on speckle patterns can require experienced sonographers and provide limited anatomical information.

These techniques can provide limited monitoring feedback on where and to what extent ultrasound engages the targeted tissues, while avoiding imaging during the active push transmit (e.g., interleaved or post-push acquisitions) to mitigate ultrasound interference, which can lead to missing temporal information of ultrasound effects when FUS is being applied. Furthermore, certain FUS techniques do not provide both axial steering and real-time capabilities for optimizing acoustic parameters for tissue engagement.

Therefore, there is a need for FUS techniques and systems for stimulating target tissue and simultaneous imaging of the stimulation with improved confidence in FUS-induced effects.

### SUMMARY

The disclosed subject matter provides techniques for monitoring and/or modulating target tissue using ultrasound. The disclosed subject matter provides systems and methods for simultaneous monitoring and modulating target tissue. The disclosed subject matter also provides methods for mitigating pain in a subject.

In certain embodiments, the system for simultaneous monitoring and modulating target tissue can include a focused ultrasound (FUS) stimulation probe for stimulating the target tissue, an imaging probe for obtaining ultrasound images of displacement on the target tissue, and a processor configured to provide an image of target tissue within about 2 seconds from the stimulating. In non-limiting embodiments, the imaging probe and the FUS stimulation probe can be coaligned. The FUS stimulation probe can include a first central frequency range, and the imaging probe can include a second central frequency range. In non-limiting embodiments, the first and second frequency ranges are different.

In certain embodiments, the disclosed system can include an adaptive holder. The imaging probe can be inserted through a central opening of the FUS stimulation probe and be coaligned through the adaptive holder.

In certain embodiments, the FUS stimulation probe can be a single element FUS probe. In non-limiting embodiments, the FUS stimulation probe can be configured to generate positive pressures up to 30 MPa. In some embodiments, the FUS stimulation probe can be configured to generate a stimulation pulse. The pulse duration of the stimulation pulse can range from about 0.5 ms to about 10 ms. In non-limiting embodiments, the first central frequency range can be from about 1 MHz to 4 MHz. In some embodiments, the FUS stimulation probe can induce the displacement of target tissue without damaging the target tissue.

In certain embodiments, the second central frequency range can be from about 4 MHz to about 16 MHz.

In certain embodiments, the processor can be further configured to obtain RF data from ultrasound images, conduct delay-and-sum beamforming, and generate a displacement map of the target tissue through a 1D cross-correlation.

In certain embodiments, the target tissue can be a nerve, a brain, a heart, or a combination thereof.

In certain embodiments, the disclosed subject matter provides a method for simultaneous monitoring and modulating target tissue. The method can include modulating the target tissue by inducing displacement with a single ultrasound acquisition device, acquiring ultrasound images of the target tissue using the single ultrasound acquisition device, and generating an image of the displacement within 2 seconds from the modulating. The single ultrasound acquisition device can include a focused ultrasound (FUS) stimulation probe and an imaging probe that are coaligned.

In certain embodiments, the method can further include obtaining RF data from acquired ultrasound images, conducting delay-and-sum beamforming, and generating a displacement map of the target tissue through a 1D cross-correlation.

In certain embodiments, the method can further include adjusting ultrasonic parameters of the single ultrasound acquisition device. In non-limiting embodiments, the ultrasonic parameters can include a pulse duration of the FUS stimulation probe, a pulse sequence of the FUS stimulation probe and the imaging probe, a total time-of-flight (TOF), a pressure level, a central frequency of the FUS stimulation probe, a central frequency of the imaging probe, or combinations thereof. In some embodiments, the pressure level can range from about 5 MPa to about 9 MPa. In non-limiting embodiments, the pulse duration of the FUS stimulation probe can range from about 0.5 ms to about 10 ms. In some embodiments, the central frequency of the FUS stimulation probe can range from about 1 MHz to 4 MHz, and the central frequency of the imaging probe can range from about 4 MHz to about 16 MHz.

The disclosed subject matter provides a method for mitigating pain in a subject. The method can include identifying the target nerve, applying focused ultrasound (FUS) to the target nerve, and inducing displacement between about 1 micron to about 60 microns of the target nerve.

In certain embodiments, the method can further include acquiring ultrasound images of the target nerve and generating an image of the displacement within 2 seconds from the applying FUS to the target nerve.

In certain embodiments, the target nerve is stimulated with a pressure generated by FUS. In non-limiting embodiments, a level of the pressure can be up to about 8 MPa. In some embodiments, the displacement can be induced without damaging the target nerve.

The disclosed subject matter will be further described below.

### BRIEF DESCRIPTION OF THE DRAWINGS

FIG. 1 provides schematics of an example thermal-FUS ultrasound system.

FIG. 2A provides an example ultrasound transducer setup for neuromodulation of sciatic nerves. FIG. 2B provides example designs of the disclosed transducer system with an imaging transducer holder.

FIG. 3A provides a diagram depicting the waveforms used to drive the FUS transducer.

FIG. 3B provides a graph showing hydrophone measurements of the FUS transducer's pressure distribution.

FIG. 4A provides a diagram of the tilted imaging plane waves for simultaneous imaging and stimulation pulse sequences during displacement imaging. FIG. 4B provides images showing output frame captures during displacement imaging for a 4 MPa (MI=1.6) pulse (top) and interframe displacement between compound frames (bottom). FIG. 4C provides a graph showing representative displacement traces over varying FUS pulse lengths.

FIG. 5A provides a diagram showing mouse leg topology and relative locations of the stimulation and recording sites. FIG. 5B provides an example displacement imaging. FIG. 5C provides a graph showing single compound muscle action potentials (CMAPs) from a single FUS stimulus. FIG. 5D provides a graph showing traces of multiple CMAPs from a single FUS stimulus.

FIG. 6 provides a parameter space map of average cumulative nerve displacement over pulse duration and pressure (top) and graphs showing FUS parameters that induced CMAPs (bottom).

FIG. 7A provides a waterfall plot of example electromyography (EMG) traces acquired at increasing FUS focus depths with and without CMAP. FIG. 7B provides displacement maps showing interframe tissue displacement at subsequent focal depths corresponding to traces in FIG. 7A.

FIG. 8 provides a graph showing the correlation of evoked CMAP amplitude vs. average interframe nerve displacements.

FIG. 9A provides histological H&E staining images for 100 pulses of sham ultrasound, 22 MPa, and 30 MPa at the same spot with a PRF of 0.3 Hz. FIG. 9B provides 2D Temperature heatmaps showing spatial temperature changes from a single pulse of FUS. FIG. 3C provides boxplots showing a change of temperature for certain pressures. FIG. 9D provides graphs showing results from gait analysis −1, 1, and 5 days from FUS sonication on the left hind limb.

FIG. 10 provides an image showing the contribution of cavitation (top) and a graph showing measured cavitation at the sciatic nerve over a single trial (bottom).

FIG. 11 provides a graph showing an example EMG artifact corruption in pulses 5 ms and longer in both EMG traces.

FIG. 12 provides an example FUS-imaging system in accordance with the disclosed subject matter (top), compounded plane-wave B-mode image (bottom left), and displacement tracking image in real-time (bottom right).

FIG. 13 provides a graph showing within-FUS pulse sequence as recorded using a hydrophone in free-field and a flow diagram of a single ultrasound data acquisition sequence.

FIG. 14 provides images showing interframe displacement maps (top) and interframe displacement traces after decimation and corresponding SNR calculations over pressures between 0.7 to 11 MPa peak negative (bottom).

FIG. 15 provides a graph showing log plots of DAS computational time of CPU, parallel CPU, MATLAB GPU parallel computing toolbox, and CUDA GPU over RF length.

FIG. 16 provides a graph showing computational times for CPU, parallel CPU, MATLAB parallel GPU, and CUDA GPU operations for increasing interpolated grid sizes for DAS beamforming.

FIG. 17 provides displacement maps produced using a gelatin tissue-mimicking phantom at −5, 0, 5, and 10 mm focal depths.

FIG. 18 provides images showing tracked displacements before, during, and after FUS push in the human subject forearms.

FIG. 19 provides an image showing targeting and monitoring FUS neuromodulation using simultaneous displacement imaging (left) and graphs showing average and standard deviation interframe and cumulative displacement traces (right).

FIG. 20 provides a graph showing thermal ratings for heat pulses with FUS and sham treatment to the median nerve.

FIG. 21 provides a displacement map for a FUS pulse where the nerve was located 5 mm laterally from the FUS focus (off-target) and the interframe displacement at the ROI on the nerve for on-vs. off-target stimulations and corresponding thermal ratings for each trace (right).

FIG. 22 provides a graph showing data of pain ratings for 13 human subjects.

FIG. 23 provides a graph showing average ratings between FUS and sham.

FIG. 24 provides schematics of an example clinical FUS neuromodulation system.

FIG. 25 provides an image of an exemplary FUS stimulation system and graphs showing concurrent electrical signals.

FIG. 26 provides schematics and graphs showing transcutaneous electrical stimulation to the median nerve around the wrist.

FIG. 27 provides graphs showing skin flare response in humans over time using laser Doppler perfusion flowmetry.

FIG. 28 provides graphs showing Pain rating vs. displacement of FUS stimulation on the median nerve (Left) and 2D distribution of pain ratings and their corresponding FUS-induced nerve displacement (Right).

FIG. 29 provides a graph showing pain rating averages from ratings collected in the first half vs. the second half show no difference.

It is to be understood that both the foregoing general description and the following detailed description are exemplary and are intended to provide further explanation of the disclosed subject matter.

### DETAILED DESCRIPTION

The disclosed subject matter provides techniques for monitoring and/or modulating target tissue using ultrasound. The disclosed subject matter provides systems and methods for simultaneous monitoring and modulating target tissue using focused ultrasound (FUS). The disclosed subject matter can be used to mitigate pain in a subject. The disclosed techniques provide real-time feedback of the FUS beam used and improved safety for tissue stimulation.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

As used herein, “treatment” or “treating” refers to inhibiting the progression of a disease or disorder, or delaying the onset of a disease or disorder, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect can be prophylactic in terms of completely or partially preventing a disease or condition or a symptom thereof and/or can be therapeutic in terms of a partial or complete cure for a disease or disorder and/or adverse effect attributable to the disease or disorder. “Treatment,” as used herein, covers any treatment of a disease or disorder in an animal or mammal, such as a human, and includes: decreasing the risk of death due to the disease; preventing the disease or disorder from occurring in a subject which can be predisposed to the disease but has not yet been diagnosed as having it; inhibiting the disease or disorder, i.e., arresting its development (e.g., reducing the rate of disease progression); and relieving the disease, i.e., causing regression of the disease.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc. In certain embodiments, the subject is a pediatric patient. In certain embodiments, the subject is an adult patient.

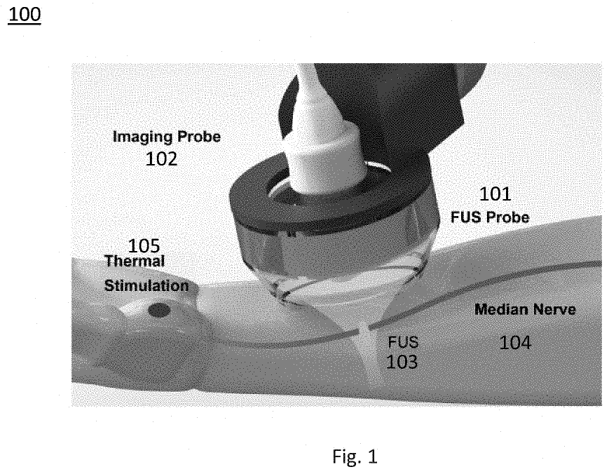

In certain embodiments, the disclosed subject matter provides a system for simultaneous monitoring and modulating target tissue. As shown in FIG. 1, an example system 100 can include a FUS stimulation probe 101, an imaging probe 102, and a processor. In non-limiting embodiments, the FUS stimulation prob 101 and the imaging probe 102 can be coaligned for simultaneous stimulation and monitoring of the target tissue (FIG. 1).

In certain embodiments, the disclosed system can include a FUS stimulation probe for stimulating target tissue. The FUS stimulation probe can generate an acoustic radiation force and induce displacement of the target tissue during the FUS modulation pulse. For example, the FUS stimulation can generate displacement on the target tissue without damaging the target tissue. The displacement can range from about 1 μm to about 300 μm, from about 1 μm to about 250 μm, from about 1 μm to about 200 μm, from about 1 μm to about 150 μm, from about 1 μm to about 100 μm, from about 1 μm to about 50 μm, from about 1 μm to about 40 μm, from about 1 μm to about 30 μm, from about 1 μm to about 20 μm, from about 1 μm to about 10 μm, from about 1 μm to about 5 μm, or from about 1 μm to about 3 μm.

The FUS transducer can be flat or concave, single or multi-element transducer, or an annular or sector array transducer.

In certain embodiments, the FUS stimulation probe can be set with different combinations of ultrasound parameters. The ultrasound parameters can include a peak negative pressure, a peak positive pressure, an acoustic intensity, a mechanical index, an interval inter-stimulus, a pulse duration, a duty cycle, a total time of flight (TOF), and/or a center frequency. In non-limiting embodiments, the peak negative pressure can range from about 0.5 MPa to about 11 MPa, from about 0.5 MPa to about 10 MPa, from about 0.5 MPa to about 9 MPa, from about 0.5 MPa to about 8 MPa, from about 0.5 MPa to about 7 MPa, from about 0.5 MPa to about 6 MPa, from about 0.5 MPa to about 5 MPa, from about 0.5 MPa to about 4 MPa, from about 0.5 MPa to about 3 MPa, from about 0.5 MPa to about 2 MPa, or from about 0.5 MPa to about 1 MPa. In non-limiting embodiments, the peak positive pressure can range from about 1 MPa to about 30 MPa, 1 MPa to about 25 MPa, 1 MPa to about 20 MPa, 1 MPa to about 15 MPa, 1 MPa to about 10 MPa, or 1 MPa to about 5 MPa. In non-limiting embodiments, the pulse duration can be about 10 milliseconds (ms), about 9 ms, about 8 ms, about 7 ms, about 6 ms, about 5 ms, about 4 ms, about 3 ms, about 2 ms, about 1 ms, or about 0.5 ms. In some embodiments, the pulse duration can be from about 0.5 ms to 10 ms. In non-limiting embodiments, the center frequency of the FUS stimulation probe can range from about 1 megahertz (MHz) to about 4 MHz, from about 1 MHz to about 3 MHz, or from about 1 MHz to about 2 MHz. In some embodiments, the center frequency of the FUS stimulation probe can be about 1.1 MHz. Certain parameters (e.g., acoustic intensity, mechanical index, and interval interstimulus) can be derated from the aforementioned parameters (i.e., peak negative pressure, peak positive pressure, and duty cycle).

In certain embodiments, the range of the duty cycle can be from about 0% to about 100%, from about 5% to about 100%, from about 10% to about 100%, from about 15% to about 100%, from about 20% to about 100%, from about 25% to about 100%, from about 30% to about 100%, from about 35% to about 100%, from about 40% to about 100%, from about 45% to about 100%, or from about 50% to about 100%. In non-limiting embodiments, the disclosed system can stimulate the targeted region with duty cycles 0-100% and pulse durations from about 0.5 ms to about 5 ms.

In certain embodiments, the ultrasound parameters can be pre-programmed and/or adjusted depending on the target tissue or subject. For example, a single-element FUS stimulation probe with about 4 MHz center frequency, about 1 ms of pulse duration, and a pressure level from about 4 MPa to 30 MPa can be used to activate a sciatic nerve of mouse. In non-limiting embodiments, a 4-element FUS stimulation probe with about 1.1 MHz center frequency, about 5 ms of pulse duration, and a pressure level from about 1 MPa to about 8 MPa can be used for stimulating a median nerve of a human subject.

In certain embodiments, the disclosed system can include an imaging probe. The imaging probe can be used for obtaining ultrasound images of the target tissue. The imaging probe can also be used for locating target tissue, monitoring stimulation/modulation of the target tissue, and confirming delivery FUS to the target tissue. For example, the imaging probe can perform B-mode ultrasound-guided imaging displacement of the target tissue before/after the FUS stimulation. In non-limiting embodiments, the imaging probe can be linear, curved, phased, 1D, or 2D array with a number of elements varying from 32 to 1024 elements.

In certain embodiments, the imaging probe can have at least one ultrasonic parameter. The ultrasound parameter can include pulse duration, a duty cycle, a total time of flight (TOF), and/or a center frequency. In non-limiting embodiments, the ultrasonic parameter can be customized or modified based on the FUS stimulation probe and/or target tissue. For example, the center frequency of the imaging probe can range from about 4 megahertz (MHz) to about 16 MHz, from about 4 MHz to about 15 MHz, from about 4 MHz to about 14 MHz, from about 4 MHz to about 13 MHz, from about 4 MHz to about 12 MHz, from about 4 MHz to about 11 MHz, from about 4 MHz to about 10 MHz, from about 4 MHz to about 9 MHz, from about 4 MHz to about 8 MHz, from about 4 MHz to about 7 MHz, from about 4 MHz to about 6 MHz, or from about 4 MHz to about 5 MHz. In some embodiments, the center frequency of the imaging probe can be about 7.8 MHz. In non-limiting embodiments, the center frequency of the imaging probe can be different from the center frequency of the FUS stimulation probe for simultaneous stimulation and monitoring. For example, as both imaging and stimulation transducers can be driven simultaneously, the center frequencies can be chosen to be as separate as possible for reducing FUS interference. In non-limiting embodiments, imaging pulse durations can include multiple cycles, for example, 2 cycles.

In certain embodiments, the disclosed system can perform simultaneous imaging of tissue displacement during FUS stimulation. The disclosed system can perform within-pulse imaging across the imaging and stimulation probes without interleaving sequences. In order to utilize both probes without interleaving, the pulse duration of the FUS probe can be selected to a proportion of the total time-of-flight (TOF) for a wave to travel from the imaging probe face to a scatterer at the edge of the imaging window and back. For example, to generate an image at 41 mm from the transducer face (e.g., the aperture of 9.94 mm), the TOF (e.g., 71 μs) can set the extended FUS burst to be an integer multiple of the TOF. The TOF can be determined based on imaging frame rate, which can be set to any parameters that the imaging transducer/probe can achieve (e.g., less than 1 ms of TOF for a frame rate of 1000 fps/PRF). In non-limiting embodiments, the imaging probe can track micron-sized displacements using frame rates determined by the calculated TOF, without interleaving the FUS pulses and imaging acquisition. The total frame rate of the technique can be target depth dependent. In non-limiting embodiments, the frame rate can be higher than 1000 frames per second (fps)/pulse repetition frequency (pro. For example, the frame-rate can be about 14 kHz for a depth of 41 mm.

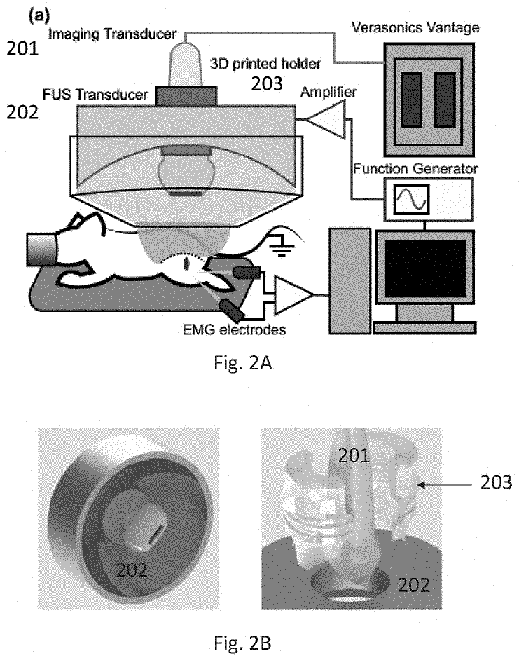

In certain embodiments, the disclosed system can further include an adaptive holder. As shown in FIG. 2A, the imaging probe 201 and the FUS stimulation probe 202 can be coaligned through the adaptive holder 203. For example, the imaging probe can be inserted through a central opening of the FUS stimulation probe through the adaptive holder 203 (FIG. 2B). In non-limiting embodiments, the adaptive holder 203 can be customized based on the structure of the FUS and imaging transducers. For example, the adaptive holder can be designed in a Cad program and printed using a 3D printer. The adaptive holder can include resin, plastic, metal, or combinations thereof. The adaptive holder can position the focus of the FUS transducer within the imaging plane of the imaging transducer. For example, the focus of the FUS transducer can be centered within the imaging plane of the imaging transducer.

In certain embodiments, the disclosed system can include a processor coupled to the FUS stimulation probe and/or the imaging probe. In non-limiting embodiments, the processor can be coupled to the probes directly (e.g., wire connection or installation into the probes) or indirectly (e.g., wireless connection). The processor can be configured to perform the instructions specified by software stored in a hard drive, a removable storage medium, or any other storage media. The software can include computer codes, which can be written in a variety of languages, e.g., MATLAB and/or Microsoft Visual C++. Additionally or alternatively, the processor can include hardware logic, such as logic implemented in an application-specific integrated circuit (ASIC). The processor can be configured to control one or more of the system components described above. For example, and as embodied herein, the processor can be configured to control imaging and ultrasound stimulation. Additionally, or alternatively, the processor can be configured to control the output of the function generator and/or the transducer to provide the FUS to the subject.

In certain embodiments, the processor can be configured to provide an image of target tissue before/after the FUS stimulation. For example, after stacking, transferring, and/or obtaining radio frequency (RF) imaging frames, the processor can beamform the frames and images using a delay-and-sum (DAS) algorithm. In non-limiting embodiments, the processor can perform parallel calculations using a graphic processing unit. In some embodiments, the processor can filter the beamformed RF data using a filter (e.g., comb notch filter) at the predetermined frequency. For example, the predetermined frequency can include fundamental (e.g., 1.1 MHz) and harmonic frequencies (e.g., 2.2-12.1 MHz) of the FUS stimulation probe. In non-limiting embodiments, the processor can calculate the displacement of the target tissue by performing 1D cross-correlation, which can include an estimate of the correlation between two signals acquired during different time points in the same region in the FUS stimulated tissue. The cross-correlation estimation provides how much the tissue is displaced in comparison to a prior time. The processor can further generate displacement maps based on the RF data and the calculated displacement of the target tissue. The processor can provide the displacement maps to a user. For example, the displacement map can be smoothed using a 2D median filter (e.g., 0.2% of the reconstruction grid), and displacements can be overlaid onto the filtered B-mode images after calculation of frames and displayed in real-time.

In certain embodiments, the processor can be configured to provide an image of the target tissue and/or displacement maps within the predetermined time. The predetermined time can be less than about 2 seconds, about 1 second, about 500 milliseconds (ms), about 250 ms, about 100 ms, about 50 ms, about 25 ms, about 10 ms, about 5 ms, about 2 ms, or about 1 ms from the stimulation of the target tissue. For example, the processor can reduce the processing time by implementing the DAS beamforming algorithm into GPU kernels. Variables used to beamform in real-time can be stored in the shared memory on the GPU before imaging execution. All delays can be calculated and summed up for each pixel in the desired linear interpolation grid. 3D indexing can be used for each calculation in time (e.g., frames) and spatial (e.g., depth and lateral) points. In non-limiting embodiments, beamforming operation can be performed by passing the raw RF data as a singular vector array.

In certain embodiments, the target tissue can be any tissues. For example, the target tissue can be a nerve, a brain, a heart, muscle, tendons, ligaments, skin, vessels, or a combination thereof.

In certain embodiments, the disclosed system can induce action potentials from the muscle of a subject by stimulating target tissue using FUS without damages. The disclosed system can record the activation of the subsequent compound muscle action potential (CMAP). For example, the disclosed system can include electrodes to perform electromyography. One electrode can be grounded, and the other electrodes can be placed into the muscle of the subject. In non-limiting embodiments, the disclosed system can record the amplitude and/or waveform of CAMP.

In certain embodiments, the disclosed subject matter provides a method for simultaneous monitoring and modulating target tissue. An example method can include modulating target tissue by inducing displacement with a single ultrasound acquisition device, acquiring ultrasound images of the target tissue using the single ultrasound acquisition device, and generate an image of the displacement within 2 seconds from the modulating. In non-limiting embodiments, the single ultrasound acquisition device can include a focused ultrasound (FUS) stimulation probe and an imaging probe that are coaligned. For example, the imaging probe can be inserted through a central opening of the FUS stimulation probe through the adaptive holder. The adaptive holder can be customized based on the structure of the FUS and imaging transducers.

In certain embodiments, the method can further include adjusting ultrasonic parameters of the single ultrasound acquisition device. The ultrasonic parameters can include a pulse duration of the FUS stimulation probe, a pulse sequence of the FUS stimulation probe and the imaging probe, a total time-of-flight (TOF), a pressure level, a central frequency of the FUS stimulation probe, a central frequency of the imaging probe, or combinations thereof. In non-limiting embodiments, the ultrasonic parameters can be adjusted for the target tissue. For example, the single ultrasonic acquisition device can include a single-element FUS stimulation probe with about 4 MHz center frequency, about 1 ms of pulse duration, and a pressure level from about 4 MPa to 30 MPa for activating a sciatic nerve of mouse and a 128-element linear array with 16 MHz center frequency for imaging the activation of the sciatic nerve (e.g., displacement). In some embodiments, the single ultrasonic acquisition device can include a 4-element FUS stimulation probe with about 1.1 MHz center frequency, about 5 ms of pulse duration, and a pressure level from about 1 MPa to about 8 MPa can be used for stimulating a median nerve of a human subject and an imaging probe with about 7.8 MHz center frequency. In some embodiments, the center frequency of the imaging/FUS probe can be adjusted to be different from the center frequency of the FUS/imaging stimulation probe for simultaneous stimulation and monitoring. For example, as both imaging and stimulation transducers can be driven simultaneously, the center frequencies can be chosen to be as separate as possible for reducing FUS interference.

In certain embodiments, the method can further include obtaining RF data from acquired ultrasound images, conducting delay-and-sum beamforming, and generate a displacement map of the target tissue through 1D cross-correlation. For example, after stacking, transferring, and/or obtaining radio frequency (RF) imaging frames from the ultrasonic acquisition device, the frames and images can be beamformed using a delay-and-sum (DAS) algorithm. The beamformed RF data can be filtered using a filter (e.g., comb notch filter). In non-limiting embodiments, the displacement of the target can be calculated by performing 1D cross-correlation on the beamformed RF data. The calculated displacement can be used for generating displacement maps. In non-limiting embodiments, the displacement map can be smoothed using a 2D median filter (e.g., 0.2% of the reconstruction grid), and overlaid onto the filtered B-mode images after for clear location and visualization of the stimulation.

In certain embodiments, the disclosed subject matter provides a method for mitigating pain in a subject. An example method for mitigating pain can include identifying the target nerve, applying focused ultrasound (FUS) to the target nerve, and inducing displacement between about 1 to about 60 microns of the target nerve.

In certain embodiments, the target nerve can be identified using an imaging probe. For example, the imaging probe can be configured to perform B-mode imaging to locate the target nerve. In non-limiting embodiments, the target nerve can be any motor or sensory or mixed nerves in the peripheral nervous system, for example, a median nerve, a radial nerve, sural nerve, a vagus nerve, or a sciatic nerve.

In certain embodiments, the FUS with at least one ultrasound parameter can be applied to the target nerve. The FUS can induce displacement of the target nerve to mitigate pain in a subject. For example, the FUS can induce displacement of the target nerve from about 1 micrometer (μm) to about 60 μm. The applied FUS can induce the predetermined displacement without damaging the target tissue (e.g., no full/partial ablation). In non-limiting embodiments, the ultrasound parameter can include a peak negative pressure, a peak positive pressure, a pulse duration, a duty cycle, a total time of flight (TOF), and/or a center frequency. For example, the 1.1 MHz, 4-annular array FUS stimulation probe with axially focusing of the beam (e.g., 1×15 mm) can be used to stimulate the median nerve up to positive pressures of about 8 MPa for mitigating pain.

In certain embodiments, the method can further include acquiring ultrasound images of the target nerve and generating an image of the displacement within 2 seconds from the applying FUS to the target nerve. For example, ultrasound images of the target nerve can be obtained using an imaging probe for confirming the delivery of FUS to the target nerve. B-mode images and RF data can be obtained using the imaging probe. For example, a 7.8 MHz imaging probe can be coaligned inside the center opening of the FUS transducer and used for simultaneously obtaining the B-mode images and RF data during the FUS stimulation. In non-limiting embodiments, the obtained ultrasound images and data can be used for generating an image of the displacement of the target nerve. For example, after obtaining RF imaging frames of the target nerve before/after the FUS stimulation from the imaging probe, the frames and images can be beamformed using a DAS algorithm. The displacement of the target can be calculated by performing 1D cross-correlation on the beamformed RF data. The calculated displacement can be used for generating displacement maps. In non-limiting embodiments, the displacement map can be overlaid onto the B-mode images of the target nerve for clear location and visualization of the stimulation.

The disclosed subject matter can reduce sensations of a subject to pain. For example, delivery and targeting of FUS with the disclosed parameters to nerved of a subject can reduce sensation to nociceptive thermal pain. In non-limiting embodiments, the disclosed techniques can be used to induce less than about 60 microns, less than about 50 microns, less than about 40 microns, less than about 30 microns, less than about 25 microns, less than about 20 microns, less than about 15 microns, less than about 10 microns, less than about 5 microns, or less than about 1 micron of displacement for the reduction in pain without damaging tissue/nerve. In some embodiments, the disclosed techniques can be used to induce about 28 microns of displacement for the reduction in pain.

### EXAMPLES

### Example 1: Displacement Imaging for Focused Ultrasound Peripheral Nerve Neuromodulation

Ultrasound neuromodulation system: the experimental setup in FIG. 2A was used to activate the sciatic nerve in anesthetized mice. Two commercially available ultrasound transducers were used in a confocally aligned configuration: A FUS stimulation transducer (H-215, 4 MHz, single-element FUS; Sonic Concepts Inc., Bothell, Wash., USA) and an imaging array (L22-14vX-LF, 16 MHz, 128 elements linear array; Vermon, Tours, France). The imaging transducer was inserted through a central opening in the FUS transducer and was coaligned using a 3D-printed attachment through a central opening in the FUS transducer and with the faces of both transducers 15 mm apart. The attachment was designed in a CAD program (Solidworks; Dassault Systemes, Waltham, Mass., USA) and printed in clear resin in a 3D printer (Form2; Formlabs, Somerville, Mass., USA) using the direct dimensions of the imaging transducer provided by the manufacturer (FIG. 2B). The part puts the focus of the FUS transducer within the imaging plane of the imaging transducer. Since both transducers are driven simultaneously, the center frequencies were chosen to be as separate as possible, reducing FUS interference. A function generator (33220a; Keysight Tech., Santa Rosa, Calif., USA) amplified by a 150 W RF power amplifier (A150; E&I, Rochester, N.Y., USA) drove the FUS transducer. Imaging transmits and receive events were acquired using a research ultrasound system (Vantage 256; Verasonics Inc., Redmond, Wash., USA) research platform. Ultrasound was transmitted through a coupling cone filled with degassed water and degassed ultrasound gel coupled to the upper thigh of the mouse

Animal preparation: Male C57BL/6J mice, weighing between 22 g to 28 g, were used in all experiments (n=6). Male mice were used to decrease variability between animals. No nerve excitability differences were observed between male or female mice. Mice were anesthetized with isoflurane: 4% during induction and preparation, 2%. Physiological saline (0.1 mL per 10 g of body weight) was subcutaneously injected every 1-2 h to prevent dehydration. Mouse hind limbs were shaved and dehaired using a depilatory cream. An infrared heating pad was used to maintain proper body temperature (36.5° C.) throughout all experiments (FIG. 2). The mouse was placed in a pronated orientation so that the sciatic nerve ran superficially below the skin.

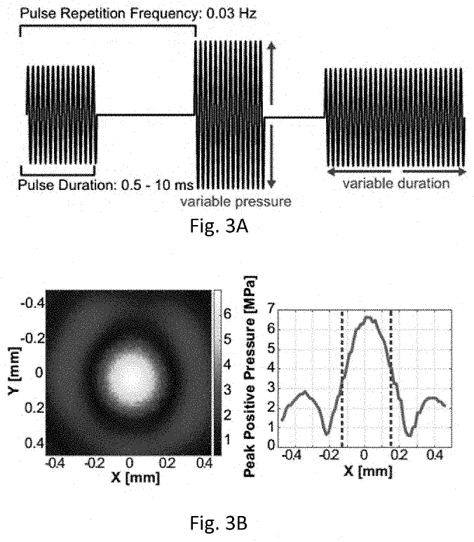

Acoustic parameters: The acoustic parameters that were varied were acoustic pressure and pulse duration. The acoustic pressure was varied from 4 to 30 MPa in steps of 4 MPa, and the pulse duration was varied from 0.5 to 10 ms in steps of 0.5 ms. These ranges were established based on the success rate and safety analysis. In addition, histological and behavioral analyses were performed to check for potential damage, specifically using the setup and parameters. Single pulses were emitted at 0.03 Hz to mitigate cumulative bioeffects in and around the nerve. These parameters are summarized in FIG. 3A. Parameter space exploration was done using constant 1 ms pulses using the whole pressure range and constant 24 MPa pressures using the whole pulse duration range.

Hydrophone measurements: Hydrophone measurements were conducted to characterize the FUS beam in free field degassed water. A fiber-optic hydrophone (HFO-690, Onda Corp, Sunnyvale, Calif., USA) was positioned on a 3D manipulator, and the FUS transducer was held stationary. Lateral and axial beam profiles were achieved at 6.5 MPa, showing that the FUS focal size is 0.24 by 1.19 mm full width half maximum (FWHM) (FIG. 3B). Pressure curves were acquired by sweeping the whole input voltage range for 10 cycles (sufficient to ramp up to saturated pressure). A second capsule hydrophone (HLG-0200, Onda Corp, Sunnyvale, Calif., USA) was used to characterize pressures under 5 MPa. The fiber optic curve was fit to the capsule hydrophone results to generate the whole pressure range.

Electromyography recordings: Electromyography was performed using two bi-polar needle electrodes (EL451; Biopac, Goleta, Calif., USA) grounded to either the loose skin on the back of the neck, the table, or the tail. One electrode was placed 1 mm into the tibialis anterior and the other 1 mm into the gastrocnemius muscle. The head was fixed in a stereotaxic frame, and the legs were immobilized to reduce movement artifacts in the EMGs. The mouse was then placed in a custom-built faraday cage to eliminate external noise sources from the recording electrodes. A two-second window surrounding the FUS trigger was recorded to capture any CMAP activation.

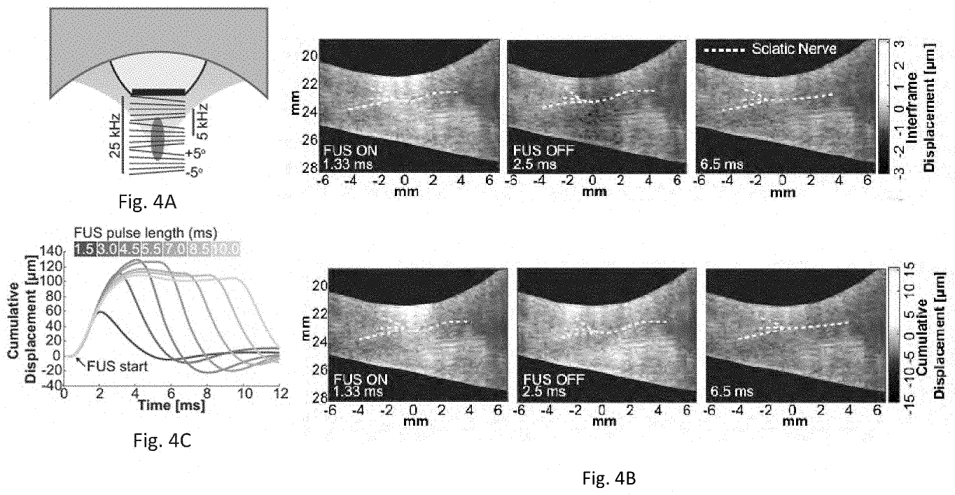

Displacement imaging: Displacement imaging was performed by synchronization of the FUS pulse and the imaging sequence (FIG. 4A). The imaging transducer sequence connected to the Verasonics triggered the function generator so that plane waves were sent 0.5 ms before to 0.5 ms after the FUS pulse. Five sequential plane wave transmits were tilted from −50 to +50 and summed up to produce a compounded image with higher SNR. After summation, the compounded frame rate was 5 kHz, and this was used for imaging tissue movement before, during, and after FUS sonication. Notch filters were designed to remove FUS interference from the fundamental and sequential harmonics. Since the FUS pulse is 1 ms, having 5 angles allows 5 fully compounded frames for displacement estimation. Increasing the number of angles improves B-mode quality but decreases the amount of frames within the pulse, thus displacement quality is reduced. Angles less than 50 were found to be more susceptible to FUS interference.

After acquisition, delay-and-sum beamforming maps were calculated using the CUDA API for real-time processing on a GPU (Tesla K40, Nvidia, Santa Clara, Calif., USA). The delay calculations were parallelized onto 3 grids of 1024 threads, specific to the GPU. 1D normalized cross-correlation was performed on RF sampled at 4 points per wavelength and calculated also using GPU processing. Correlation window length of 9λ and a 95% overlap provided adequate balance between processing speed and accuracy of displacement in real-time. Interframe displacement movies were generated and immediately displayed to visualize how FUS engages the nerve during each modulation event in ˜300 ms. FIG. 4B shows frame captures of interframe displacement and its summation (cumulative) over the course of one FUS sonication in the mouse leg during (1.33 ms), immediately after (2.5 ms), and long after FUS (6.5 ms). The FUS pulse was turned on at 1 ms and off at 2 ms. Characteristic displacement traces at the nerve for various pulse lengths can be seen in FIG. 4C. The resolution of our displacement imaging technique is 96.25 microns, the wavelength of the imaging transducer. In addition, regarding the displacement sensitivity, the Cramer-Rao Lower Bound (CRLB) of the cross-correlation technique with our transducer specifications was calculated. The lower limit of the displacements we can measure for 1 dB signal-to-noise (SNR) and 0.5 correlation coefficient (experimentally obtained during 24 MPa FUS pulses) is 0.897 microns.

FUS targeting of the sciatic nerve: The FUS transducer was positioned using a 3D motorized positioner (Velmex, Bloomfield, N.Y., USA). Landmarks such as the femur and the trifurcation branching of the sciatic nerve into the sural, femoral, and tibial nerves were used as visual indicators of the location of the sciatic nerve. FUS at 1-5 MPa was used as a targeting pulse to gently perturb the nerve. Resultant tissue interframe displacement was estimated and displayed in real-time to validate placement of the FUS focus. Minor adjustments could then be made before the start of an experiment.

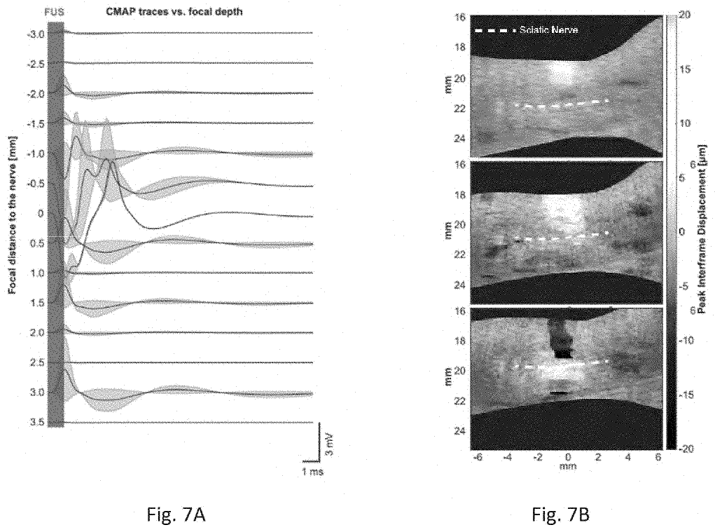

The disclosed subject matter was used to measure the effect of radiation force on sciatic nerve activation in two separate experiments. The disclosed subject matter was used to characterize nerve displacements and muscle activations over a wide parameter space. The CMAP waveform and nerve displacement from each sonication were recorded. The disclosed subject matter also was used to determine whether displacement is a prerequisite for neuromodulation. The focus was placed at the top of the skin and rastered downwards past the sciatic nerve. Interframe displacement maps and CMAP amplitudes were measured for each pulse. Sonications that did not elicit muscle contraction are presented in FIG. 7A, but they were excluded from CMAP amplitude vs. pressure, duration, and inter-frame displacement analysis.

Data analysis: Parameter space maps were generated by measuring average interframe nerve displacement using an ROI (1 mm×0.5 mm) at the center of the focus and nerve. 50 displacement images per parameter (8 pressures×10 PD parameters) per sciatic nerve (n=6) were acquired. Displacements were excluded when higher sonication pressures created noise that could not be properly filtered. Parameter space maps were interpolated using a cubic spline interpolation. All statistics were run using GraphPad Prism 7.04. For correlation experiments (CMAP energy vs interframe displacement), a non-parametric Spearman correlation was run to compute the R-value between interframe displacement measurements and CMAP energy. For gait analysis, a two-way ANOVA with multiple comparisons was used to evaluate sciatic nerve function before and after sonication

Histological analysis: A separate experiment in mice was conducted to demonstrate safety parameters of FUS (n=6 nerves). Mice were anesthetized and 100 pulses of FUS at 0.2 Hz pulse repetition frequency (PRF) to the sciatic nerve in the same area was applied. Nerves received 1 of 5 experimental parameters and 1 sham sonication. Sonications were applied with a PRF of 0.2 Hz for 50 seconds using a subset of parameters where CMAPs were observed (22, 24, 26, 28, and 30 MPa; 1 ms pulse duration). Sciatic nerves were immediately dissected post-sacrifice and perfused with 4% PFA and 70% EtOH for 3 days before sectioning and Hematoxylin and Eosin (H&E) Staining. Red blood cell extravasation, degenerated myelin, cell apoptosis, inflammation and swelling, and protein degradation were used as indication of damage.

Safety assessment of FUS parameters: Temperature measurements of single 1 ms FUS stimulations at 22 MPa and 28 MPa were acquired using a needle thermo-couple in heterogeneous chicken muscle to mimic muscle and nerve temperature. 2D raster scans were performed in both axial and lateral directions of the FUS beam. Temperature distributions of peak temperature rise show that temperature is spatially confined to twice the focal volume. A sharp 3-dB temperature decrease occurred at 0.4 mm away from the center of the focus laterally and 1.6 mm in depth. The temperature returned to baseline within 2 seconds after 1 ms stimulations.

Functional safety was conducted using gait analysis (Cat-Walk XT; Nodulus, Utrecht, Netherlands) 1 day before, 1 day after, and 5 days after sonication (n=10 male; 5 sham, 5 FUS). 10 FUS sonications and displacement imaging pulses (24 MPa, 1 ms, 30 s interstimulus interval) were applied to the left hind leg in anesthetized mice. Function of the sciatic nerve was analyzed using measurements of the sciatic function index (SFI), the max contact mean intensity of the left hind paw (values between 0-255) and the measured paw print length. SFI was calculated using:

$$SFI = 118.9 \times \frac{T S E - T S N}{T S N} - 51.2 \times \frac{P L E - P L N}{P L N} - 7.5 \qquad ( 1 )$$

PL is the print length, TS is the toe spread, ITS is the intermediate tow spread. Subscripts E and N indicate experimental and normal contralateral hind paws, respectively.

Preliminary cavitation Mapping: A separate follow-up experiment was conducted to examine if and where cavitation occurs using the parameters in experiment 2. The sciatic nerve was located as before, but the nerve experienced 147 pulses in the same position. Cavitation using cavitation mapping and CMAPs were recorded simultaneously. 147 sonications were applied to the nerve in the same location. The same system described above can be used to map cavitation without any additional hardware. Cavitation maps were generated using a receive-only (passive) acquisition scheme. The imaging transducer received passive emissions from the FUS transmission to form an image so that cavitation mapping occurs during the FUS pulse. The receive signals were temporally delayed based on the geometry of the transducer and the propagation times for each element (delay, sum, and integrate beamforming). Then the power cavitation image was log-compressed relative to the maximum pixel intensity. The cavitation signals that occurred during the 1 ms pulse duration were integrated to generate a single cavitation image. Acoustic cavitation emissions from stable cavitation were extracted by selecting for ultra-harmonic frequencies (relative to 4 MHz) within the bandwidth of the imaging transducer. The resultant cavitation image for stable cavitation was overlaid onto a B-mode image of the leg. An ROI of similar size to the focal beam was chosen to quantify cavitation for each FUS stimulation and the normalized intensity of the integrated signal was plotted for each of the 147 sonications.

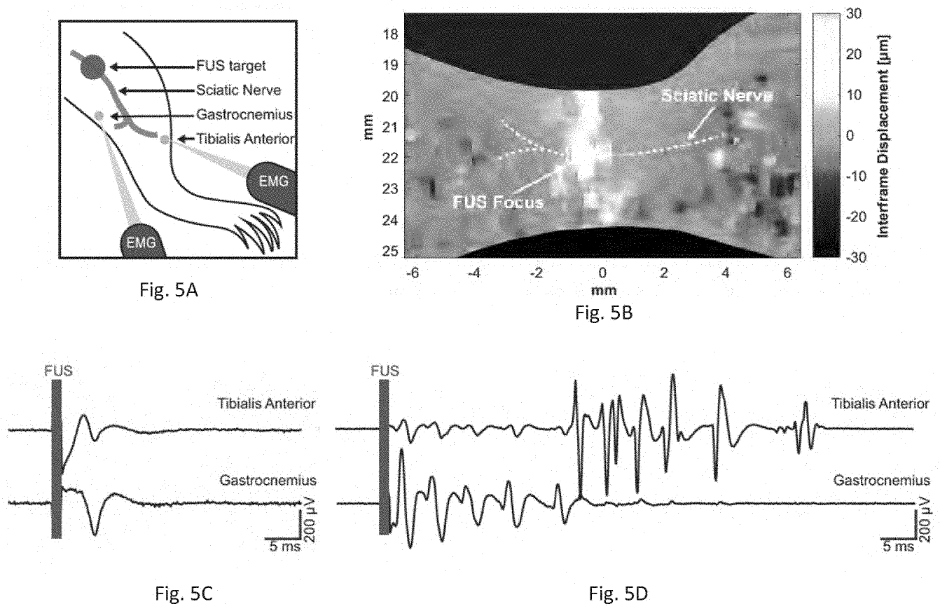

Displacement imaging can target and monitor nerves for neuromodulation: Using the technique developed for simultaneous modulation and imaging, the FUS focus was visualized via displaced tissue at the sciatic nerve and measure corresponding CMAPs. FIGS. 5A-5D summarizes the targeting of the sciatic nerve and acquisition of both displacement and CMAP waveforms. FUS was initially applied upstream of EMG electrodes inserted in muscles innervated by the sciatic nerve (FIG. 5A). Short pulses of 1 ms, 1-5 MPa peak positive FUS was delivered and simultaneously imaged to visualize wave propagation. FIG. 5B shows maximum downward interframe displacement at the sciatic nerve, validating FUS positioning for subsequent experiments. Using higher pressure FUS (24-28 MPa), CMAPs can be elicited using similar 1 ms pulses (Supplemental Video S2). FIGS. 5C and 5D show representative EMG traces in both muscle groups. A majority of FUS-evoked events result in a single muscle contraction (FIG. 5C). However, multiple muscle contractions, such as the one shown in FIG. 5D, were also observed from a single pulse.

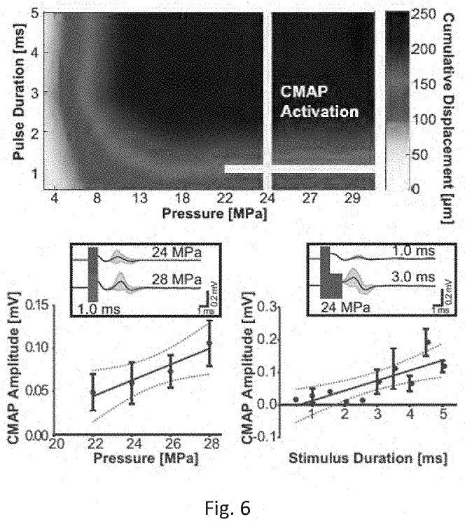

The amount of displacement at the nerve over both acoustic pressure and pulse duration parameters were recorded. FIG. 6 shows a parameter space map summarizing cumulative displacement amplitudes found in the leg. The parameters where CMAPs were observed are indicated by two perpendicular lines. CMAPs are observed for higher pressures but over the whole pulse duration sweep. Higher pressures are stronger indicators of activation than the length of the pulse duration. Only single muscle activation events by FUS were used in this analysis. By holding pulse duration constant at 1 ms, increases in pressure seem to linearly increase the amplitude of responses (R2=0.9255, p=0.038). CMAP probability for these parameters are summarized in Table I and II.

TABLE I
Probability of success for CMAP activation over pressure.

| Pressure [MPa] | probability [%] |
|---|---|
| 22 | 8 |
| 24 | 8 |
| 26 | 44 |
| 28 | 40 |

TABLE I
Probability of success for CMAP activation over pulse duration.

| PD [ms] | probability [%] |
|---|---|
| 0.5 | 4 |
| 1.0 | 10 |
| 1.5 | 4 |
| 2.0 | 2 |
| 2.5 | 6 |
| 3.0 | 12 |
| 3.5 | 14 |
| 4.0 | 18 |
| 4.5 | 26 |
| 5.0 | 28 |

Cumulative nerve displacement at this pressure range varied from 140 to 180 μm. By holding pressure constant (24 MPa), increases in pulse duration also linearly increased corresponding CMAP amplitudes (R2=0.6361, p=0.0057). Example traces shown in FIG. 6 subplots show average waveform changes in muscle activation. As pulse duration increases, the electrical interference artifact starts to impede proper EMG analysis. Thus, pulses longer than 5 ms were not included in the EMG analysis despite their success in generating spiking activity.

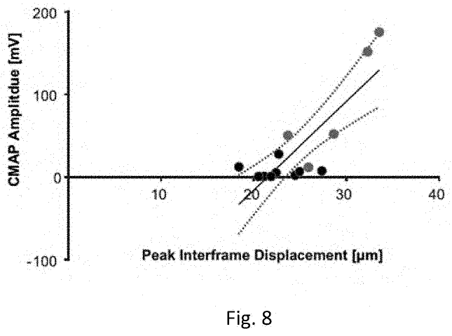

CMAP activity from mouse sciatic nerve is associated with acoustic radiation force: To determine if there is a correlation between CMAP and tissue displacement, the FUS focal position was varied to achieve varying degrees of nerve displacement. The FUS focus was moved from the upper region (starting at the skin) to the bottom of the mouse leg (n=4) in the supine position, covering a distance of approximately 7 mm. Nerve displacements were measured using a region-of-interest (ROI) surrounding the nerve over the focal depth (FIG. 7A). The location of the nerve (denoted as 0 mm) was 3.5 mm below the surface of the skin. CMAP activations were elicited within ±1 mm around the sciatic nerve, coincident with the FUS focal spot size, with a probability of 30%±20%. The step size was chosen to overlap with half the FUS FWHM focal area so that the nerve was subjected to maximum pressure. Displacement maps corroborate the focus position at each depth and nerve displacement measurements were recorded (FIG. 7B). Measurements of 29.1 μm (±0.5 μm, STD) to 34.4 μm (±0.2 μm, STD) of peak interframe displacement were shown to elicit CMAP activity. Furthermore, the EMG amplitude and the peak interframe displacement of the nerve incurred during modulation were found to be well correlated (R2=0.6791, p=0.0094, (FIG. 8)). The lowest interframe nerve displacement required for an elicited CMAP amplitude was 18.7 μm while the probability of successful activation proportionally increased with the total nerve displacement.

Certain pressures were derated for attenuation in muscle tissue using the following equation:

α+α₀f^{1.18} (2)

In Equation 2, α0 is 3.3 dBcm⁻¹ MHz⁻¹ through skeletal muscle (3 mm mouse), and f is the center frequency of the FUS in MHz. The peak negative pressures range up to 13 MPa in mice.

Pulse durations up to 5 ms, which is about 67% longer than the pulse duration that caused a plateau in displacement at 3 ms (FIG. 4C). Pulses longer than 3 ms were found to corrupt the EMG signal since the artifact from FUS stimulation overlaps with the CMAP trace.

The disclosed subject matter provides a method that can validate FUS targeting and provide mechanistic insight into the underpinnings of peripheral neuromodulation. The results show that high frame-rate displacement tracking during short FUS pulses can visualize focal displacements in the mouse leg. Using this technique, through radiation force parametric space exploration, i.e., varying both pulse duration and pressure, clear correlation with CMAP amplitude was shown. Nerves experiencing inter-frame displacements above 18 μm were more likely to result in CMAP generation. The amplitude of CMAPs increasing with nerve displacements provides evidence towards the hypothesis that ultrasound neuromodulation is driven by nerve deflection as a result of the highly focused acoustic radiation force. Therefore, the results show that this method can be a tool for informed targeting and mechanistic monitoring of FUS neuromodulation. This technique can prevent off-target effects and raise confidence in future FUS neuromodulation in nerves and even the brain.

Micron-sized displacements using high frame-rate compounded plane wave imaging before, during, and after FUS excitation pulses can be displayed back in real-time for real-time adjustments. The sensitivity of the disclosed technique to noninvasively image and localize minute displacements (<5 μm at 3 MPa within 1 ms) in vivo provides unique capability of real-time monitoring of both the mechanism and successful FUS targeting and modulation at safe acoustic levels. Since the beam being imaged is the same as the stimulation FUS, the same potential effects on wave propagation such as aberration, interference, attenuation, and/or scattering can also be monitored. Moreover, the CMAP amplitudes indicate that using an imaging pulse during the FUS does not affect neuromodulation output. Therefore, monitoring with mechanical imaging constitutes a critical safety tool for mitigating unintentional modulation in surrounding tissue regions (e.g., blood vessels or tendons) while focusing in the intended region and optimizing the required acoustic intensity for neuromodulation. Other methods for displacement imaging during neuromodulation often require longer ultrasound pulses to engage tissues at detectable displacement ranges. The disclosed subject matter can provide an effective metric for noninvasive FUS targeting in vivo and determine specific conclusions about CMAP activation. For example, acoustic pressure can have an influence on CMAP probability more than pulse duration. Using the technique, longer pulse durations can be plateaued the amount of displacement during the pulse. This saturation can explain why increases in pressure affected CMAPs more than pulse durations. Sciatic nerves were chosen because they are mixed nerve bundles with both sensory and motor fibers. Though sensory activation is more relevant to neuromodulatory therapeutics, having motor neurons allows sensory activation in mice to be interpreted through CMAP recordings in EMG. Since increased CMAP amplitudes are a result of increased recruitment of nerve fibers, correlations show that increased deflection of the nerve can contribute to more motor nerve recruitment. Moreover, due to motor nerves responding to mV level voltages, the high pressures used cab be necessary to mediate mechanotransduction of the nerve to these levels. As a result, the disclosed subject matter provides the unique capability of using the actual FUS neurostimulation pulse to qualitatively target and monitor nerve engagement during sonication. FUS-induced excitation was observed in brain circuits and nerves. However, the inhibitory effects were also shown. Neuro-modulation can be achieved using pulsed ultrasound, but can both increase or not decrease action potential probability with continuous wave and increases in acoustic intensity. This disagreement between observations can be a result of inaccurate and/or blind targeting without feedback that FUS was delivered correctly. The utility of imaging the radiation force generated during FUS stimulation of the peripheral nerves can be a method for visualizing FUS propagation during the whole neuromodulation sequence. Using the same pulse, nerves in mice were stimulated and relay information, in real-time, regarding the breadth and locality of tissue engagement by FUS.

The increased nerve deflection can induce higher levels of nerve recruitment and can be mediated through increases in both FUS pressure and duration. A 1 ms and 24 MPa FUS stimulus was employed as a base parameter to limit thermal effects from ultrasound and increase the likelihood of CMAP activation. The range of pressures and pulse durations used were determined based data showing short durations, higher pressure FUS bursts increased stimulation success. A 4 MHz transducer with a 1.19×0.24 mm focal size was employed, which can explain the need for higher pressures to achieve the same stimulation success. Increases in pressure engage additional volumes of tissue, thus raising the probability of modulating the nerve, which can compensate for targeting precision, out-of-plane positioning (elevation direction) with such a small focus. The disclosed system utilized an unprecedented spatial specificity (0.2 mm versus several mm, i.e., above 1 mm) and reached targeting accuracy far beyond other systems, as no targeting confirmation was observed.

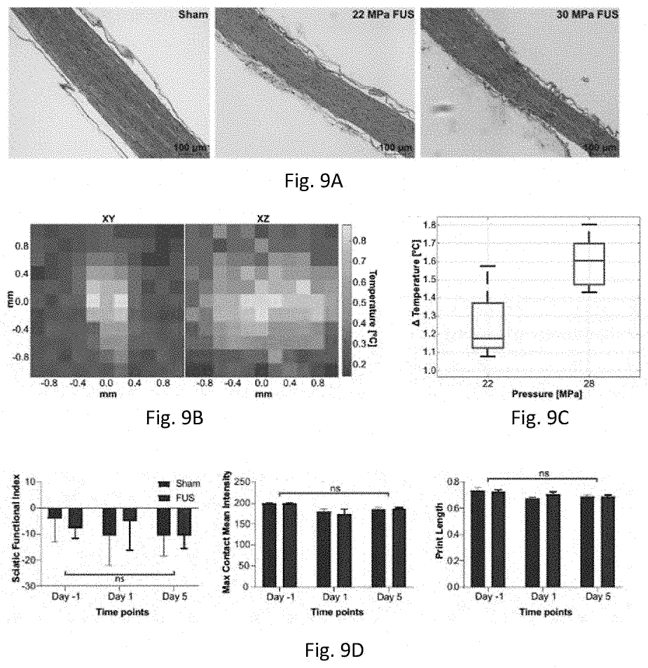

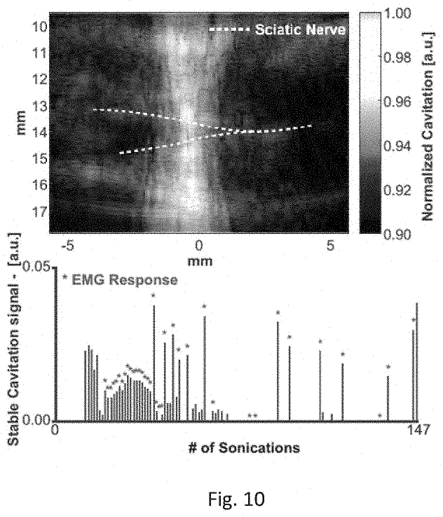

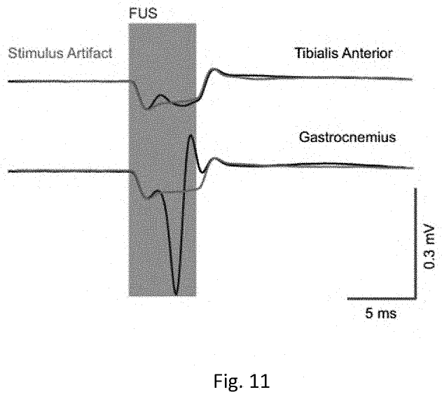

CMAPs were observed at the whole range of pulse durations given a sufficiently high pressure. This can indicate that the physical stretch of the nerve from FUS can trigger CMAP events, especially given evidence of established links between mechanical forces and neural function and activity. Regarding the safety of the disclosed technique, H&E stains in FIG. 9A show that sonications at all parameters explored appear safe and do not show apparent damage compared to sham sonications. There is no red blood cell extravasation or myelin disruption characteristic of damage to the sciatic nerve. Regarding the temperature elevations from the parameters used, a significant increase was not observed in the local temperature generated. Using a needle thermocouple in ex vivo chicken breast, the temperature elevation of the parameters was measured (FIG. 9B-9C). 2D temperature maps show the spatial temperature elevation around the FUS focus. The maximum local temperature measured was 1.6° C. (±0.1° C., STD) and 1.3° C. (±0.2° C., STD) at the highest and lowest parameters, respectively. Although temperatures returned to baseline at most 2 seconds after the stimulus, the interstimulus interval between FUS application in this study was set to 30 seconds to prevent the accumulation of heat.

The low temperature induced by the disclosed FUS is unlikely to generate nerve activation. To further validate the safety of our technique, gait analysis was conducted using the CatWalk XT program to analyze left hind limbs of mice undergoing FUS (n=5) and sham (n=5) sonications. Trials were acquired −1, 1, and 5 days from FUS sonication, where 10 stimulations were applied to the hind limb of each anesthetized mouse (24 MPa, 1 ms, 30 s interstimulus interval). A two-way ANOVA (FIG. 9D) shows no significant difference in the sciatic functional index (SFI) (p=0.4491), the max contact mean pixel intensity (p=0.8841), and the print length (p=0.2442) between sham and FUS mice. A test for multiple comparisons shows that there is a significant difference in day −1 and day 1 in the max contact mean intensity in FUS mice (p=0.0153), which also occurred between day −1 and days 1 and 5 in sham mice. These differences can be a result of anesthetizing and shaving only the left hind limb on day 0. Moreover, the SFI across all time points (used for measuring the direct function of the sciatic nerve) was comparable to SFI values in normal, healthy populations (−4.3±17.3), indicating that our FUS sonications did not functionally impair mice sciatic nerves. One of the differences between the disclosed subject matter and other ARFI, MR-ARFI, and shear wave techniques can be the ability to image and measure transient displacement during FUS delivery (<1 ms), whereas all other techniques image after the application of radiation force or has temporal resolutions inadequate for these fast pulses.

Displacement-based nerve imaging was developed to noninvasively target and monitor neuromodulation of mouse motor nerves in vivo. Micron-sized displacements were mapped in the nerve and surrounding regions, and the correlation between displacement and activation amplitude provides evidence towards the contribution of acoustic radiation force for activating nerves. The disclosed system can perform cavitation mapping for the same FUS pulse without additional hardware. Displacement amplitude thresholds for successful FUS modulation can provide a metric for consistent and reproducible FUS modulation at safe acoustic levels. Towards novel therapeutics for pain, the disclosed subject matter can provide objective measurements for the evaluation and efficacy of these treatments.

### Example 2: Displacement Imaging During Focused Ultrasound Median Nerve Modulation: Human Pain Sensation Mitigation

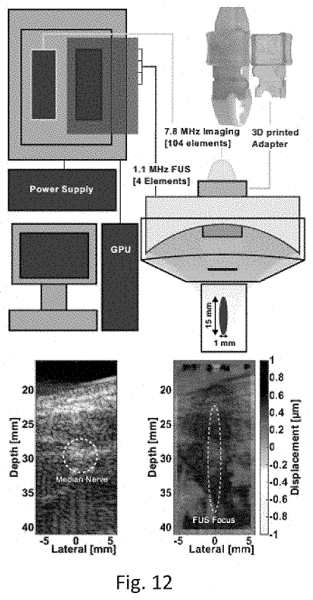

Example systems: The DAQ system includes one 256 channel Vantage research platform with the HIFU option (Verasonics, Kirkland, Wash., USA) coupled with the extended burst power supply FIG. 12. The power supply enables driving multiple cycle transmits. Half of the 256 connectors were connected to a 104 element P12-5 Phased Array transducer (ATL Philips, Bothell, Wash., USA) for simultaneous imaging and displacement tracking. The other 128 elements were connected to an RF matching box for a 4 element, 1.1 MHz FUS transducer (SonicConcepts, Bothell, Wash., USA). A customized matching box directs power from the 64 channels in the right connector to the 4 element annular rings (16 mm width) with area (450, 550, 650, and 70 mm2); each annular element is driven by 16 channels. The FUS transducer has an active diameter of 64 mm with a 40 mm opening where the imaging transducer was placed through (FIG. 12). The attached coupling cone opening is 77 mm in diameter with two tubes for degassing the water in the transducer cone or inflation/deflation of the membrane. The focus of the FUS transducer (0.5×15 mm) was aligned using a custom 3D printed mold so that the plane of the imaging transducer was centered at 30 mm in depth. The FUS frequency and size of the focal region were chosen to optimize adequate radiation pressure, relative to the focal size-to-nerve ratio. The focus encompasses the nerve diameter completely in the axial direction and 20% in the lateral direction. RF signals acquired for HFR displacement tracking were processed in real-time using a GPU CUDA-accelerated delay-and-sum (DAS) beamforming and 1D cross-correlation algorithm.

Compounded plane-wave images were used for the initial targeting of the nerve. 5 angles (±9°) and 1 transmit-receive operation per angle were used to generate a B-mode image (FIG. 12) with the focus at 30 mm. Beamformed RF data was fed into a normalized cross-correlation algorithm to generate displacement maps of FUS pushes overlaid onto B-mode images. A 95% overlap and a window size of 12.25 mm was used to track displacements in the raw RF data. Derated peak negative pressures used in phantoms were under 10.9 MPa peak negative pressure (MI=10.1, 72.3 W/cm² ISPPA) and under 7.9 MPa (MI=7.5, 29.4 W/cm² ISPPA) in humans.

Simultaneous Single-system FUS Displacement Tracking Pulse Sequence: The disclosed subject matter can be utilized for continuous imaging and monitoring that allow displacement tracking within the FUS push. Simultaneous imaging of FUS pushes can require customized ultrasonic parameters for each transducer. A 1.5 cycle plane wave emission was programmed for all 128 channels connected to the P12-5. In order to utilize both transducers without interleaving, the pulse duration of the FUS transducer was set to a proportion of the total time-of-flight (TOF) for a wave to travel from the imaging transducer face to a scatterer at the edge of the imaging window and back. Therefore, to generate an image at 41 mm from the transducer face (aperture of 9.94 mm), the TOF (71 μs) sets the extended FUS burst to be an integer multiple of the TOF. To reach a burst pulse duration of 5 ms, matching pulse durations seen in previous PNS neuromodulation studies, 70 bursts of ultrasound without interval between pulse trains were transmitted during the FUS push. The 71 μs bursts are programmed into the right 128 channels of the DAQ, which are used to drive all four annular elements.

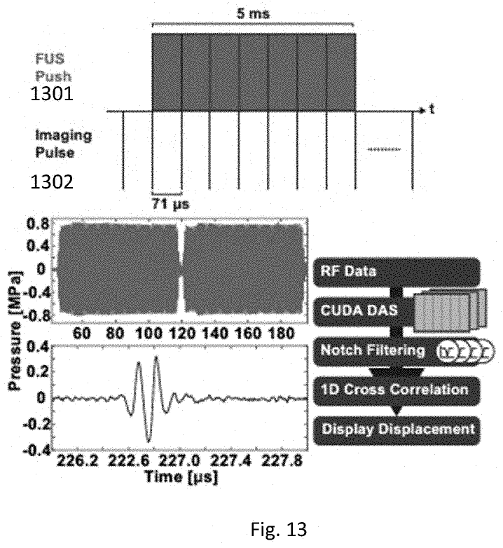

FIG. 13 shows the pulse sequence of the proposed technique. The FUS push 1301 and the imaging pulse 1302 are shown. The total frame rate of the technique is also depth dependent; for a depth of 41 mm, the frame-rate is 14 kHz. Hydrophone measurements of the FUS and imaging transducers in the free field (HGL-200 & HFO-660, Onda Corp, Sunnyvale, Calif.) is shown in FIG. 13 middle. Hydrophone measurements reveal a 17 μs gap between subsequent FUS bursts. This was caused by a combination of electronic processing time and the actuation time of the elements. The processing sequence is defined in FIG. 13. After stacking and transferring all RF imaging frames, the RF data is beamformed using a CUDA-accelerated (CUDA version 9.1) conventional DAS beamforming. The parallel calculations were performed using a GPU (Tesla k40c, Nvidia, Santa Clara, Calif., USA) with 1024 threads and 3 dimensional indexing. Beamformed RF data was filtered using a comb notch filter at all the fundamental (1.1 MHz), and harmonic frequencies (2.2-12.1 MHz) of the FUS transducer found within the P12-5 bandwidth. Harmonic Frequencies within the 60% bandwidth of the P12-5 were not applied in order to maximize signal-to-noise (SNR) of the echoes. Second-order Butterworth notch bandwidths were set to 50% of the notch frequencies, and filter coefficients were calculated before the imaging sequence so that in-sequence calculations would be devoted to beamforming and cross-correlation. Displacement calculations were performed by 1D cross-correlation with a 95% overlap and a window size of 12.25 mm. Displacement maps were smoothed using a 2D median filter of 0.2% of the reconstruction grid. Displacements were then overlaid onto the filtered B-mode images after the calculation of all frames and displayed in real-time.

CUDA benchmarking: CUDA DAS beamforming was implemented through MAT-LAB GPU kernels. Variables used to beamform in real-time were stored in the shared memory on the GPU before imaging execution. All delays were calculated and summed up for each pixel in the desired linear interpolation grid. 3D indexing was used for each calculation in time (frames) and spatial (depth and lateral) points on 1024 threads divided into 3 blocks. The kernel grid size was determined as a proportion of the reconstructed frames and spatial grid. The beamforming operation was performed by passing the raw RF data as a singular vector array.

For benchmarking GPU performance, MATLAB's GPU parallel computing toolbox, CPU multi-core (parfor), and CPU single-core operations were used as comparisons. DAS using MATLAB's toolbox was performed by pre-allocating interpolated RF samples and pre-defining forwards, backwards, and compounding delay arrays on the GPU. During imaging operation, RF data was then beamformed by linearly interpolating the RF data onto pre-indexed and pre-defined delays. The same calculations performed in C++ were separately performed on a single CPU and using parfor loops using 12 workers on two CPU cores. Two sets of analyses were performed using a sample acquired RF data of size 1152 (samples)×104 (elements). Computational time was calculated by increasing the number of samples or increasing the interpolation grid size and resolution. Benchmarking code can be made provided upon request.

Frame decimation: The accuracy and dynamic range of displacement tracking a 1.1 MHz ARF push were diminished. Therefore, to increase sensitivity and accuracy for real-time, intraprocedural targeting and monitoring, the disclosed subject matter can either increase transducer driving power or remove and decimate subsequent frames so that inter-frame displacement increases without changes to overall cumulative displacement. For every displacement map, RF of dimension 104 elements x 1152 samples were stacked by the number of frames into a larger 2D array (104×80640 for 70 frames) and acquired at the highest frame-rate required to image at a specific depth. To emphasize larger displacement estimates, we can down-sample a percentage of frames and feed the resulting beamformed RF into the displacement estimation algorithm. A factor of 0, 3, 5, 7, and 9 (i.e., removing zero, every 3rd, 5th, 7th, or 9th frame) was used for decimation. Therefore, for a 5 ms FUS pulse, the received 70 frames can be reduced to 23 frames after decimation by 3, allowing larger displacement measurements between subsequent frames. Afterward, SNR was calculated on the displacement signals for every decimation factor.

Sample preparation: a polyacrylamide tissue-mimicking phantom with 4% agar powder (Sigma-Aldrich, St. Louis, Mo.) as a scattering particle was used to validate the displacement imaging. The phantom has an elastic modulus of 10 kPa.

Human subject preparation: All human subjects (n=5) were recruited. Human subjects were seated comfortably with their forearm resting in a mechanical and movable cuff. The forearm was placed as to not introduce any physical shifting during the experiment. Degassed ultrasound gel was used to couple the transducer system bladder to the forearm. Compound B-mode imaging was used to initially place the therapeutic transducer focus on the median nerve.

Heat stimulation pulses were delivered to the C6 dermatome of the right arm (n=1) using a custom-built thermofoil device. This initial feasibility acquisition was set so that the FUS was delivered exactly when the heat signal was applied to the palm. FUS sonications (on target and off-target) were randomized within the 14 heat pulses delivered, and the subject was asked to rate the intensity of the thermal stimulus. Though the scale ranges from 0 to 10, thermal stimulation ratings were maintained in the range of 3 to 6.

Statistical analysis: Statistical testing was performed in Prism 8 (Graph-pad, San Diego, Calif.) using a non-parametric Mann-Whitney test to test for differences between ratings given concurrently with FUS or sham sonication.

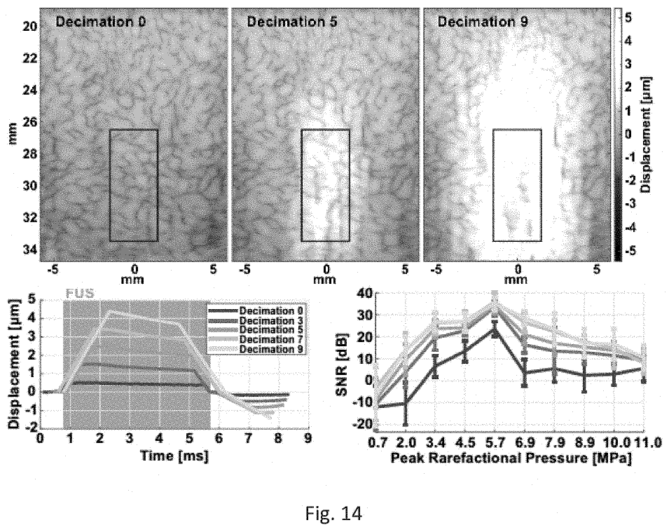

Signal-to-Noise Optimization: The imaging technique, using a 5 ms pulse sequence, in FIG. 13 was optimized by calculating SNR during the FUS pulse in a gelatin tissue-mimicking phantom for decimated displacement estimation (0, 3, 5, 7, and 9 frames) at varying levels of focal pressures (FIG. 14). The displacement maps shown are 0.16, 0.40, and 0.55 ms after triggering the FUS pulse. Displacements illustrate the ellipsoidal shape of the FUS beam but with an increased lateral extent due to decimation. All calculations were performed using the ROI defined in FIG. 14 (top), placed at the center of the transducer focus (30 mm). Ten pressure levels, 5 realizations each, were used to acquire raw RF data using the disclosed technique. Post-hoc, frames were decimated by a factor ranging from 0 to 9 frames, and the resulting displacement for each realization was estimated using normalized cross-correlation. The resulting displacement maps (FIG. 14 top) show larger interframe displacement values and illustrate the ellipsoidal focus better as more decimation is used. The associated interframe displacement traces are shown for a 5 ms FUS pulse in FIG. 14 bottom. Decimated displacements have fewer time-points within the FUS pulse but have larger displacement values. The SNR was calculated within the pulse using the following equation:

SNR=μ/σ (3)

where μ is the mean displacement and σ is the variance across all realizations within the ROI. SNR was calculated for displacement values only within the FUS pulse. Results show that, for a 5 ms pulse, decimation by 7 frames had the largest increase in SNR by 15.09±7.03 dB compared to no zero decimation across all pressure levels. As such, displacement SNR increases with pressure, peaking at 5.7 MPa until the noise from FUS harmonics severely impedes both B-mode quality and the estimated displacement. Therefore, trade-offs between maximized SNR and the number of frames within the FUS pulse indicate that optimal decimation rates can range from 3 to 7 frames. Thus, for the following study, all RF data was decimated by 5 frames before displacement estimation

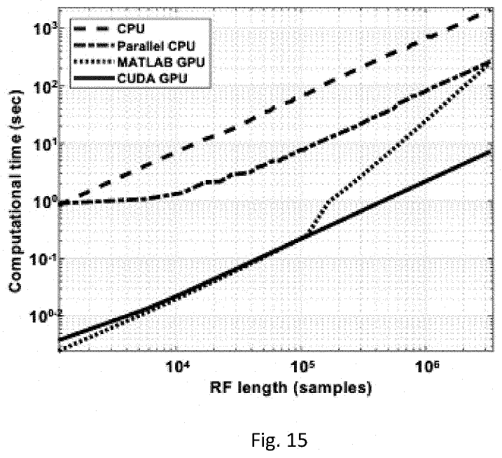

GPU Benchmarking: GPU parallelization of DAS beamforming using CUDA programming was compared to GPU parallelization using MATLAB's parallel computing toolbox, CPU parallelization (12 workers) over 2 cores, and CPU calculation on one core. FIG. 15 shows computation time over number of samples beam-formed. Averages over 3 realizations at increasing number of RF samples show similar computation time for both GPU methods until 10⁵ samples. At more than 10⁵ samples, CUDA GPU performance shows increasing computational speedup up to 35 times the MATLAB GPU computational time. CUDA GPU maintains consistent speed up over CPU and parallel CPU by 300 and 60 times, respectively. CUDA performs under real-time criteria up to 10 samples.

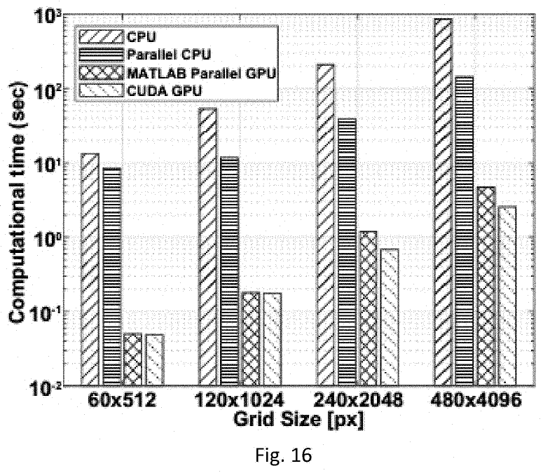

The same computational time comparisons were performed for different DAS interpolated grid size resolutions (1, 0.5, 0.25, 0.125) for 70 frames of 1152 RF samples of base grid size of 60×512 pixels. FIG. 16 shows CUDA DAS performs well compared to all other computational frameworks. The MATLAB parallel toolbox and CUDA kernel performance were similar until (240×2048 pixels) grid sizes. The CUDA kernel was not able to perform in real-time at grid sizes 480×4096 pixels and above. Benchmarking results also show considerable speedup over CPU calculations.

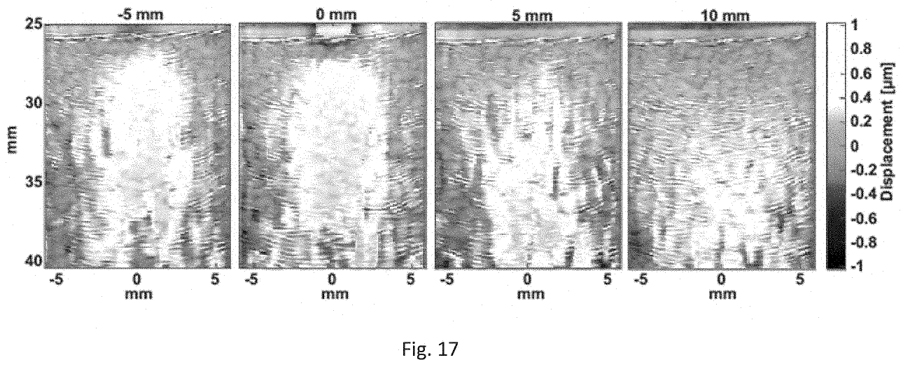

Axial Displacement Focusing: The FUS beam was electronically focused in the axial direction by calculating delays for each of the 4 annular ultrasonic elements so that the focal point is moved in the axial direction. Variations in the beam-shape occur due to the changes in the f-number. Hydrophone measurements of the beam profile in free-field show axial focusing capabilities up to ±10 mm relative to the geometric focal center. Axial focusing less than ±5 mm leads to −3 dB drop off in pressure, and greater than ±5 mm shows a greater drop off up to −6 dB. Displacement maps in a homogeneous phantom were generated for each focal depth. FIG. 17 shows relevant focal displacements at −5, 0, 5, and 10 mm focal depths relative to the geometric center at the same time point. FIG. 17 illustrates maps showing maximum inter-frame displacement, corresponding to the first frames of FUS sonication (representative displacement trace is shown in FIG. 14). The ellipsoidal shape of the focus can best be visualized at ±5 mm. At larger axial focusing positions, the displacements succumb to unfilterable FUS interference noise from overlapping FUS and imaging beams.

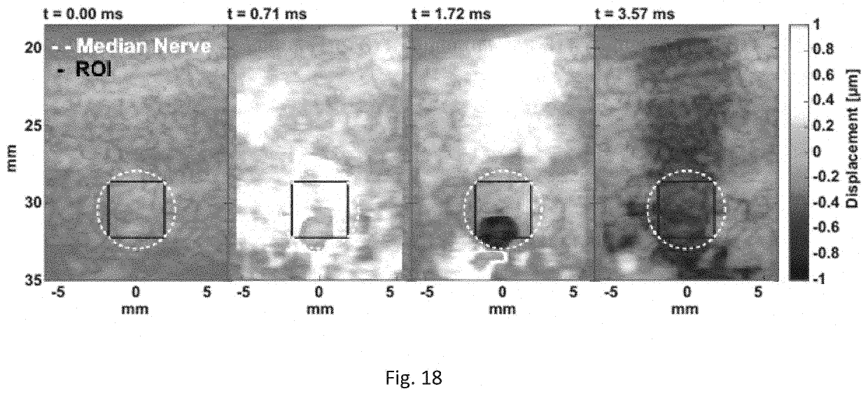

Human median nerve targeting: In FIG. 18, the pulse sequence was used to facilitate targeting for human nerve neuromodulation in healthy subjects (n=5). Nerve displacements up to 1 μm, overlapping with the FUS focus, are shown in the second frame. Upwards displacement/relaxation begins at 32 mm in frame 3 and propagates outwards in frame 4. There are visible differences in nerve displacement versus muscle (above 29 mm). This technique was used to first target the nerve, ensuring maximum FUS delivery to the nerve. Varying levels of displacement for the five subjects were measured using a 5 ms FUS pulse (5.6 MPa and 7.9 MPa peak rarefactional pressure). Mean displacement values at the center of the nerve (ROI) from 7 FUS pulses are reported in Table 3.

TABLE 3
Summary of measured displacements across subjects.

| | | Interframe | Cumulative |
|---|---|---|---|
| | Pressure | displacement | displacement |
| Subject | [MPa] | [μm] | [μm] |
| 1 | 5.6 | 2.1 ± 0.3 | 18.3 ± 2.4 |
| 2 | 5.6 | 5.5 ± 0.3 | 50.1 ± 3.4 |
| 3 | 7.9 | 4.2 ± 0.3 | 31.3 ± 2.3 |
| 4 | 7.9 | 5.2 ± 0.5 | 42.3 ± 5.5 |
| 5 | 7.9 | 5.1 ± 1.6 | 40.7 ± 10.7 |

Though the focal pressure output from the transducer was the same from subject to subject, the amount of displacement varied from 10 to 30 microns in peak cumulative displacement. After targeting validation, displacement images were used to monitor neuromodulation.

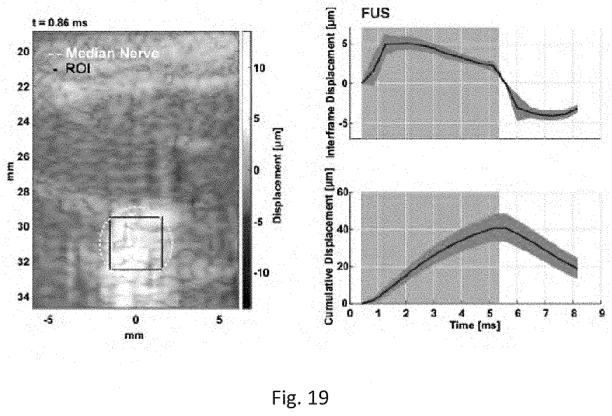

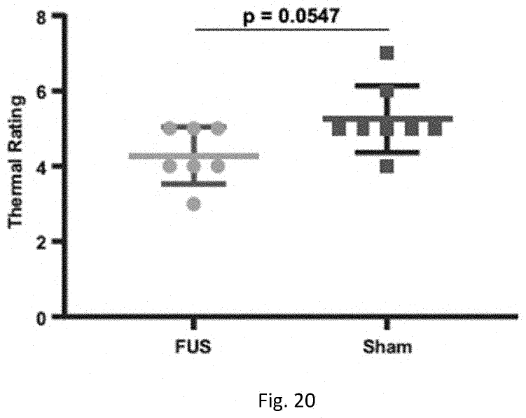

A neuromodulation experiment was conducted to validate the technique in a sensory neuromodulation experiment. Fifteen 2 second thermal pulses were delivered to the C6 dermatome of the human palm at a random interval between 3 and 4 minutes, and a subject (n=1) was asked to rate the intensity of the pulse. FUS (7.9 MPa rarefactional pressure) and sham (no FUS and off-target FUS) stimulations were randomized among the 15 heat pulses delivered in a single trial. Displacement imaging allowed monitoring of all FUS pulses and measurement of displacement at the nerve. FIG. 19 shows FUS engagement of the median nerve. To investigate acute effects of FUS on sensory perception, a pulse duration of 5 ms was chosen to ensure FUS was applied during sensory stimulus conduction; based on the average conduction velocity in a healthy human subject and the approximate distance of the FUS focus to the thermal stimulus (approximately 5-6 cm). Both interframe and cumulative displacement were estimated during the FUS pulse transmission at the peak of temperature delivery. For this particular subject, the peak interframe and cumulative displacements were estimated at the center of the nerve (black ROI) during neuromodulation was 5.1±0.7 and 40.7±7.4 microns, respectively. The maximum interframe displacement was achieved at the beginning of the pulse and the peak cumulative displacement was acquired at the end of the FUS pulse. Sources of error in the displacement curve can be due to slight movement in the subjects' arm. Preliminary data indicates that FUS can change subjective thermal perception by modulating the median nerve during heat stimulation. FIG. 20 shows ratings from heat stimuli with FUS vs. sham. A 0.9643 pain rating unit decrease, without significance, was found in FUS sonications vs. sham stimulations (p=0.0547; two-tailed unpaired Mann-Whitney test) where lower ratings were indicated for lower needle-like thermal pain.

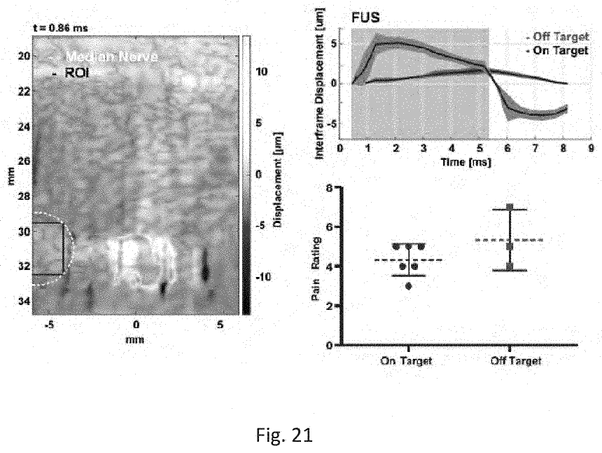

For further validation of our technique to neuromodulation, differences between FUS delivery were observed when FUS is applied directly to the nerve vs. off-target. FIG. 21 illustrates a representative displacement image when the focus was off the median nerve. FUS displacement still appears at the focus of the transducer, however the nerve was positioned 5 mm away. As a result, the displacement at the center of the nerve (ROI) had a peak cumulative displacement of 3.2±1.7 microns when the focus was off-target. Moreover, the peak interframe displacement was 1.7±0.3 microns at the end of the FUS pulse rather than the beginning as in FIG. 19 with a 5 μm difference in peaks. The thermal stimulations which were off-target had an average rating of 1 higher than when FUS was acting on the nerve directly (p=0.4524; two-tailed unpaired Mann-Whitney test).

Using a confocally aligned imaging and FUS transducer, real-time displacement tracking, mediated by CUDA-accelerated DAS, and axial focal steering using a single ultrasound DAQ system without interleaving were performed. Half of the channels can be used to drive the FUS-guided system with simultaneous custom waveforms programmed for imaging on half the channels and FUS on the other. A major advantage of using this technique, is the ability to perform RF stacking and displacement tracking in real-time. Using the Cramer-Rao lower bound for a SNR of 30 dB, a correlation coefficient of 0.98, and a window length of 11.25 μs, the disclosed technique can achieve displacements above 0.6711 um where they succumb to jitter. The disclosed transducer can achieve ±10 mm axial steering of the FUS focus. There can be a drop off in tracked displacement. This phenomenon can occur at lower positions, where these low-pressure pushes are below the noise floor. The ±5 mm range is more than adequate for targeting the median nerve in the human arm and accounting for small movements, limiting off-target effects. In the distal half of the forearm, the variation in depth of the median nerve is well within the range of 5 mm. Furthermore, since active compounded B-mode imaging is continuously used between displacement mapping sonications, the disclosed system can actively account for any movement.

Benchmarking shows the advantages of parallel GPU computing for conventional DAS compared to CPU calculations. However, the advantage of CUDA beamforming over using GPU matrices in the MATLAB toolbox are not as easily distinguishable. The computational speedup can occur at high data volumes and for displacement mapping, higher resolution interpolation grids increase SNR of displacements and can benefit from CUDA operation. This deviation can be due to how CUDA-written programs efficiently use shared memory whereas the MATLAB parallel computing toolbox does not. Other techniques, such as Doppler functional Ultra-Sound (fUS) can benefit from utilizing CUDA kernels as compounding more than 200 frames can become computationally intensive. CUDA calculation was performed in real-time, leading to less wait time and faster operation. The disclosed technique can achieve real-time specifications, and CUDA beamforming can benefit from calculating delays before initialization instead of during acquisition so that during imaging operation, other computationally intensive operations can take priority, speeding up the technique. This technique is feasible to implement in other more conventional ultrasound systems if an external FUS transducer is used or a custom device to allocate channels to a FUS transducer and imaging transducer, given the sufficient frame rates (above 10 kHz) and output power (0.5 to 1 MPa) can be achieved. A lower number of imaging channels do not necessarily impede displacement mapping, as probes such as the P4-2 (64 channels) can be used to track tissue motion. The imaging frequency can be selected so as to minimize the overlap of the FUS transducer center and harmonic frequencies.

The disclosed technique can provide the capability to facilitate targeting (n=5) of the median nerve in and neuromodulation (n=1) in healthy human subjects. Displacement maps show that the intensity of the displacement increases as the pressure is increased. Due to the heterogeneity of the forearm and boundary effects between muscle, connective tissue, and nerve fibers, the displacement map does not necessarily follow the expected ellipsoidal focus geometry. Additionally, the variance within a single subject was consistent between FUS pulses (max standard deviation of 10.7 microns). However, the disclosed technique reveals that the same output pressures do not necessarily translate to the same FUS modulation efficiency (nerve displacement) between subjects. 50 μm nerve displacement was measured from a 5.6 MPa, 5 ms pulse in 1 subject but 18 μm in another from the same pulse parameters. This can be due to a number of factors: location of the transducer on the forearm, the incident angle to the skin, tissue properties, and how well coupled the transducer system is to the skin. Preliminary findings showing FUS effects on thermal pain perception are presented. Results show that thermal pulses with coincident FUS sonication had lower thermal ratings than sham pulses. Moreover, pulses that were off-target to the nerve had a higher rating than when the focus was positioned at the center of the nerve. Displacement maps show that displacement characteristics are vastly different between on-and off-target pulses. The peak of interframe displacement was near the beginning of the pulse for on-target versus the end of the pulse for off-target (FIG. 21). FUS was capable of suppressing pain signals only when directly targeted to the nerve trunk. This effect could be explained by either the generation of afferent signals at the nerve trunk, generating a masking effect of pain, changing how the subject perceives pain, or, most likely, by the direct suppression or interruption of signals by FUS at a more proximal portion of the nerve trunk. The masking effect is unlikely to occur because the sonication using the parameters used in this study without any other stimuli did not generate any sensory response itself. The disclosed technique can adapt a single DAQ method to other elastography techniques such as harmonic motion imaging (HMI) by amplitude modulating the HIFU. Hydrophone measurements show that the disclosed system emitted a pseudo-CW push due to the electronic actuation of elements.

Real-time feedback and confirmation of targeting can lead to more conclusive results for improving the targeting. A transducer driving pressure does not necessarily lead to reproducible therapeutic levels subject to subject, providing an explanation for the wide variety of US pressures reported in neuromodulation, i.e., 1.8 MPa, 3.2 MPa, or even 50 MPa to achieve an action potential. Displacement imaging reveals real-time feedback on how much FUS is engaging the nerve, which can vary subject to subject or in the same subject depending on the coupling condition (i.e., coupling gel, incidence angle). Furthermore, the ability to focus-shift adds to the ability to modify targeting in the remedy of poor coupling, movement artifacts, and off-target effects. Variation can be detected and imaged with this technique, which can play an important role in further characterizing the mechanism of FUS neuromodulation.

By deriving a new pulse sequence and hardware combination to simultaneously drive a FUS and imaging transducer with a single ultrasound DAQ system, tracking displacements within the FUS pulse for targeting and monitoring can be accomplished. The pulse sequence and frame-rate were optimized using a homogeneous tissue-mimicking phantom and applied to in vivo human median nerve stimulation. Furthermore, the disclosed technique is able to operate in real-time and account for shifts in targeting using axial beam steering. The micron displacements in the nerve were able to confirm FUS delivery. The imaging technique presented herein was successfully validated in an experiment demonstrating FUS effects on thermal perception where the subject experienced a 0.9643 pain rating unit decrease in the needle-like pain sensation. Moreover, the disclosed technique can validate on- and off-targeting of the nerve indicating 5-micron differences at the transducer driving pressures. The imaging technique developed here is not restricted to the tissues validated, but can be applied to any ultrasound-accessible soft tissue found elsewhere in the body, such as the brain. Furthermore, the disclosed subject matter can provide real-time targeting, allowing for greater confidence in the results of FUS mechanistic studies.

### Example 3: Focused Ultrasound Median Nerve Stimulation can Modulate Nociceptive Pain

Example systems: A 1.1 MHz, 4-annular array FUS transducer (SonicConcepts, Bothell, Wash., USA) with axially focusing of the beam (1×15 mm) was used to stimulate the median nerve up to positive pressures of 8 MPa. A 7.8 MHz imaging transducer (Philips, Amsterdam, Netherlands) is coaligned inside the center opening of the FUS transducer. Both transducers were connected to a 256 channel research Vantage machine (Verasonics, Kirkland, Wash., USA) with 64 channels dedicated to the FUS and the 104 channels to the imaging transducer. The Vantage was configured with the extended burst option to drive a burst of FUS at the longer pulses required for neuromodulation. The thermal stimulator uses a MATLAB-controlled DC power supply to drive a thermistor and thermofoil (Minco, Minneapolis, Minn., USA). The example system can be seen in FIG. 1.

Methods of imaging: B-mode was first used to identify and target the median nerve in 13 healthy human subjects. Afterward, displacement imaging was used to confirm the successful delivery of FUS to the median nerve and re-position for optimal transmission. Thermal stimulation (30-50.0 for 2 seconds) was induced on the palm of the subject's hand, and FUS or sham pulses were applied for 5 ms during transmission of the pain signal to the brain. Each subject received FUS at various pressures, under 8 MPa. After each thermal pulse, the subject was asked to rate their pain on the Wong-Baker faces pain-rating scale. The subject was asked to rate the “needle-like” or “pin-prick” sensation from the thermal stimulation. Timing and protocol were controlled using an arduino UNO (Arduino LLC, Boston, Mass., USA).

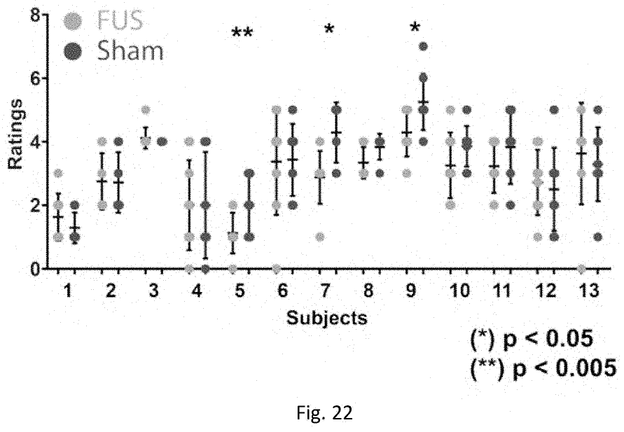

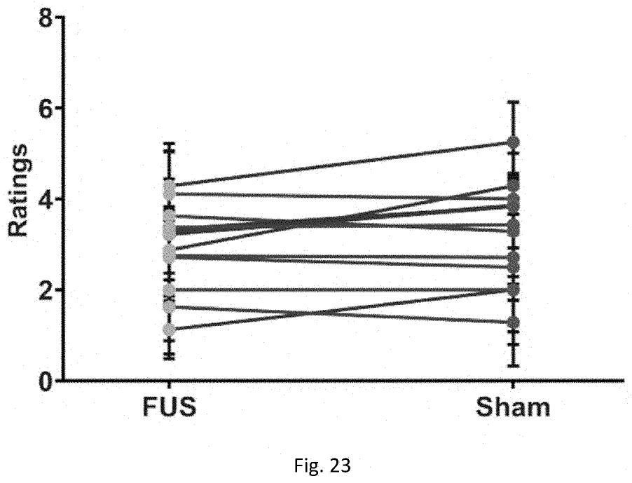

FIG. 22 shows all subjects' summary data, where FUS decreased subjective pain ratings significantly in three subjects (p<0.05 and p<0.005). Overall, 7 out of 13 subjects had decreases in pain ratings to FUS than to sham pulses (FIG. 23) for an average of 1.4 units of pain decrease. Displacements between 0 and 60 microns were measured from FUS acoustic radiation force. The disclosed imaging technique revealed that displacements above 20 microns were sufficient to reduce pain.

The reduction in pain can be due to displacement affecting the transmission of the pain signal through various fibers so that what each subject perceives can be a more dulled pain sensation. Alternatively, FUS can be interrupting transmission through some fibers where more displacement increases the number of fibers that can be affected, causing decreased pain.

The disclosed subject matter provides insight into nociceptive pain, and since the subjects were asked to rate their pain based on the “needle-like” sensation. The disclosed system can evaluate effects in faster fibers such as Aβ and large-diameter Aδ fibers and not necessarily c-fibers, which are specifically pain-sensing neurons.

Delivery and targeting of FUS to the median nerve in healthy human subjects can reduce sensations to nociceptive thermal pain. The pain was reduced in 7 out of 13 subjects with significance in three. Tracked displacement during FUS stimulation indicates that there is a threshold of displacement for the reduction in pain.

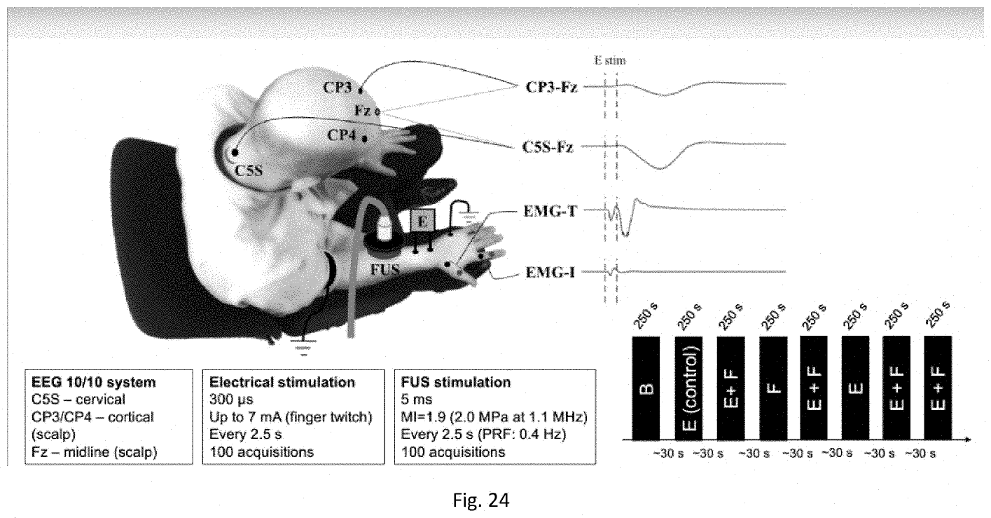

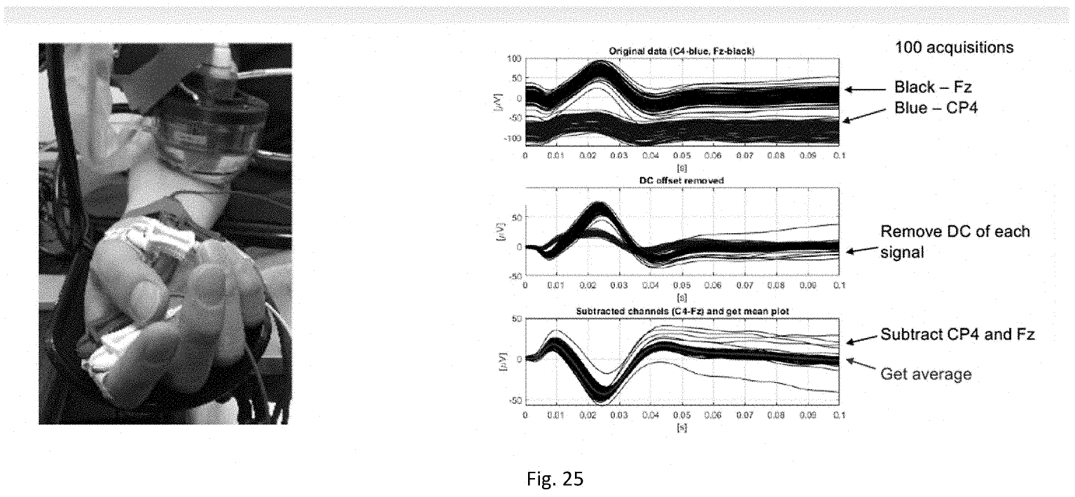

FIG. 24 shows an example clinical FUS neuromodulation system. Motor responses were evoked by electrical stimulation, while FUS was blindly switched on and off. Somatosensory evoked potential (SSEP) was recorded with electrodes at cervical and scalp locations. Multiple sequences were applied (e.g., B—baseline (no stimulation), E—electrical stimulation, E+F—electrical+FUS stimulation, and F—FUS stimulation).

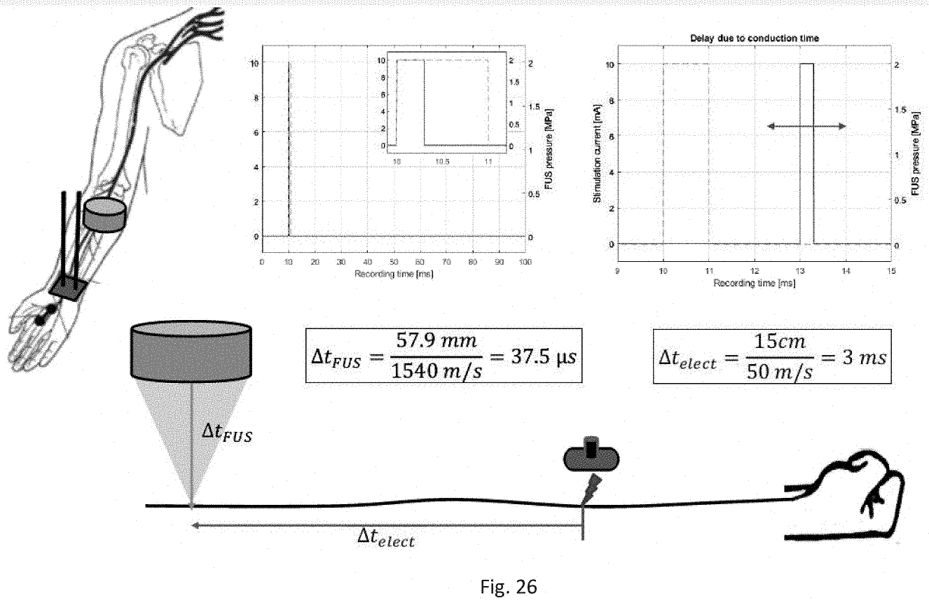

FIG. 26 shows an example FUS stimulation system and concurrent electrical signals. Subjects received transcutaneous electrical stimulation on the median nerve at the wrist region with concurrent FUS stimulation at a more proximal region of the median nerve. EMG leads were used to detect motor activity on the first 2 digits and palm, while EEG electrodes placed on the scalp and cervical regions detected SSEP signals evoked by the stimulations (see FIG. 24). The plots show the SSEP signal processing that involved 100 acquisitions, followed by DC suppression, and the averaging of signals with Fz as the reference. In non-limiting embodiments, the disclosed system can include an arm attachment for fixing the position of the subject's arm.

FIG. 26 provides schematics and graphs showing transcutaneous electrical stimulation. Transcutaneous electrical stimulation was delivered to the median nerve around the wrist, while FUS stimulation was delivered at a more proximal portion of the median nerve in the forearm. The distance between the FUS and electrical stimulation sites were approximately 15 cm, which resulted in an estimated 3 ms traveling time for the electrical stimulus to reach the FUS stimulation site (nerve conduction velocity of 50 m/s). The FUS pulse duration was set to 5 ms to allow modulating the nerve before (3 ms), during (300 μs), and after (1.7 ms) the electrical stimulus.

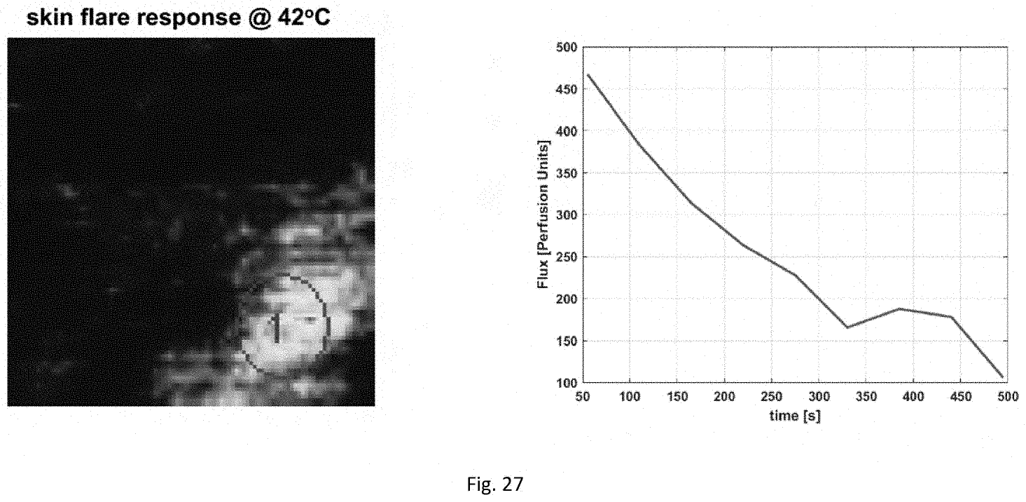

FIG. 27 provides graphs showing skin flare response in humans over time using laser Doppler perfusion flowmetry. Inflammation and peptidergic release decreased to adequate levels after 300 seconds (See FIG. 27).

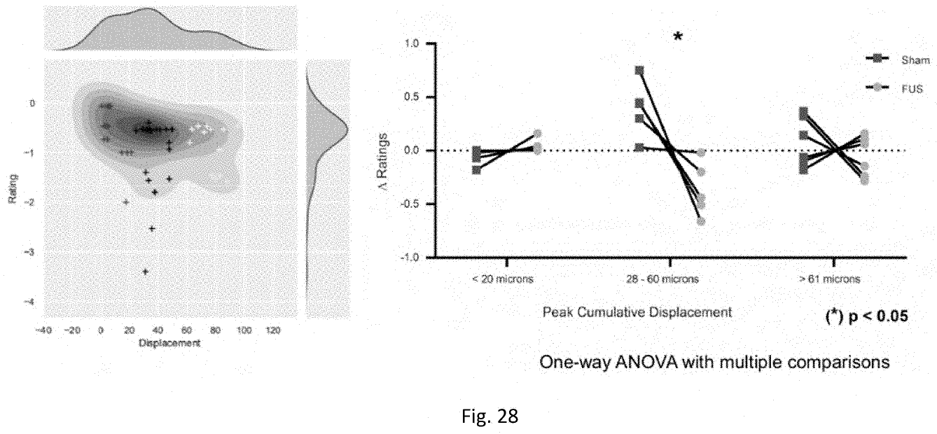

FIG. 28 provides graphs showing pain rating vs. displacement of FUS stimulation on the median nerve (Left) and 2D distribution of pain ratings and their corresponding FUS-induced nerve displacement (Right). K-means clustering revealed 3 clusters in the distribution. Changes in pain ratings in sham vs. FUS pulses separated by the 3 clusters. 1-way ANOVA with multiple comparisons shows displacements between 28 and 60 microns are significantly different.

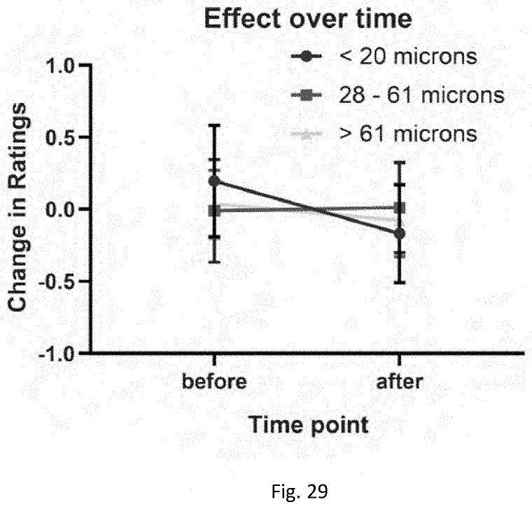

FIG. 29 provides a graph showing pain rating averages from ratings collected in the first half vs. the second half show no difference, indicating no changes in sensitivity to the thermal stimulus.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A system for simultaneous monitoring and modulating target tissue, comprising:
a focused ultrasound (FUS) stimulation probe for stimulating the target tissue, wherein the FUS stimulation probe comprises a first central frequency range;
an imaging probe for obtaining ultrasound images of displacement on the target tissue, wherein the imaging probe comprises a second central frequency range, wherein the first and second frequency ranges are different, wherein the imaging probe and the FUS stimulation probe are coaligned; and
a processor, wherein the processor is configured to provide an image of target tissue within about 2 seconds from the stimulating.

2. The system of claim 1, wherein the system further comprises an adaptive holder, wherein the imaging probe and the FUS stimulation probe are coaligned through the adaptive holder.

3. The system of claim 1, wherein the FUS stimulation probe is a single element FUS probe.

4. The system of claim 1, wherein the imaging probe is inserted through a central opening of the FUS stimulation probe.

5. The system of claim 1, wherein the FUS stimulation probe is configured to generate positive pressures up to 30 MPa.

6. The system of claim 1, wherein the FUS stimulation probe is configured to generate a stimulation pulse, wherein a pulse duration of the stimulation pulse ranges from about 0.5 ms to about 10 ms.

7. The system of claim 1, wherein the first central frequency range is from about 1 MHz to 4 MHz and the second central frequency range is from about 4 MHz to about 16 MHz.

8. The system of claim 1, wherein the target tissue comprises a nerve, a brain, a heart, or a combination thereof.

9. The system of claim 1, wherein the processor is configured to
obtain RF data from ultrasound images,
conduct delay-and-sum beamforming, and
generate a displacement map of the target tissue through a 1D cross-correlation.

10. The system of claim 1, wherein the FUS stimulation probe induces displacement of target tissue without damaging the target tissue.

11. A method for simultaneous monitoring and modulating target tissue, comprising:
modulating the target tissue by inducing displacement with a single ultrasound acquisition device, wherein the single ultrasound acquisition device comprises a focused ultrasound (FUS) stimulation probe and an imaging probe that are coaligned;
acquiring ultrasound images of the target tissue using the single ultrasound acquisition device; and
generating an image of the displacement within 2 seconds from the modulating.

12. The method of claim 11, further comprising
obtaining RF data from acquired ultrasound images;
conducting delay-and-sum beamforming; and
generating a displacement map of the target tissue through a 1D cross-correlation.

13. The method of claim 11, further comprising
adjusting ultrasonic parameters of the single ultrasound acquisition device, wherein the ultrasonic parameters comprise a pulse duration of the FUS stimulation probe, a pulse sequence of the FUS stimulation probe and the imaging probe, a total time-of-flight (TOF), a pressure level, a central frequency of the FUS stimulation probe, a central frequency of the imaging probe, or combinations thereof.

14. The method of claim 13, the pressure level ranges from about 5 MPa to about 9 MPa.

15. The method of claim 13, wherein the pulse duration of the FUS stimulation probe ranges from about 0.5 ms to about 10 ms.

16. The method of claim 11, wherein the central frequency of the FUS stimulation probe ranges from about 1 MHz to 4 MHz, and the central frequency of the imaging probe ranges from about 4 MHz to about 16 MHz.

17. A method for mitigating pain in a subject, comprising:
identifying target nerve;
applying focused ultrasound (FUS) to the target nerve; and
inducing displacement between about 1 micron to about 60 microns of the target nerve.

18. The method of claim 17, further comprising
acquiring ultrasound images of the target nerve; and
generating an image of the displacement within 2 seconds from the applying FUS to the target nerve.

19. The method of claim 17, wherein the target nerve is stimulated with a pressure generated by FUS, wherein a level of pressure is up to about 8 MPa.

20. The method of claim 17, wherein the displacement is induced without damaging the target nerve.
